(19) United States
(12) Patent Application Publication
(10) Pub. No.: US 2025/0318754 A1
(43) Pub. Date: Oct. 16, 2025

(54) SYSTEMS, DEVICES AND METHODS FOR ANALYTE SENSOR INSERTION

(71) Applicant: ABBOTT DIABETES CARE INC., Alameda, CA (US)

(72) Inventors: Vivek S. Rao, Alameda, CA (US); Louis G. Pace, San Carlos, CA (US); Hyun Cho, Berkeley, CA (US); Benjamin Jay Feldman, Berkeley, CA (US); Yi Wang, San Ramon, CA (US); Tuan Nguyen, San Jose, CA (US)

(21) Appl. No.: 19/230,772

(22) Filed: Jun. 6, 2025

Related U.S. Application Data
(63) Continuation of application No. 17/667,220, filed on Feb. 8, 2022.
Application No. 17/667,220 is a continuation of application No. 17/357,090, filed on Jun. 24, 2021, now Pat. No. 12,268,496.
Application No. 17/357,090 is a continuation of application No. 15/877,331, filed on Jan. 22, 2018, now Pat. No. 11,071,478.
(60) Provisional application No. 62/449,570, filed on Jan. 23, 2017.

(51) Int. Cl. A61B 5/145; A61B 5/00; A61B 17/34

(52) U.S. Cl. CPC A61B 5/14503; A61B 5/00; A61B 5/6849; A61B 17/3468; A61B 5/0004; A61B 5/14532; A61B 2017/347; A61B 2560/0443; A61B 2560/063

(57) ABSTRACT

Systems, devices and methods are provided for inserting at least a portion of an in vivo analyte sensor, such as a dermal sensor, for sensing an analyte level in a bodily fluid of a subject. An applicator is positioned against a skin surface and a force is applied to the applicator causing at least a portion of a sharp and an in vivo analyte sensor to be positioned in the body of the subject. In particular, disclosed herein are embodiments of applicators designed to prevent premature sharp withdrawal and/or reduce the likelihood of improper sensor insertion. Also disclosed are embodiments of applicators including sharp modules having an angled sharp which can be configured to create an insertion path for a sensor.

### CROSS-REFERENCE TO RELATED APPLICATIONS

The present application is a continuation of U.S. patent application Ser. No. 17/667,220, filed Feb. 8, 2022, which is a continuation of U.S. patent application Ser. No. 17/357,090, filed Jun. 24, 2021, now U.S. Pat. No. 12,268,496, which is a continuation of U.S. patent application Ser. No. 15/877,331, filed Jan. 22, 2018, now U.S. Pat. No. 11,071,478, which claims priority to and the benefit of U.S. Provisional Patent Application No. 62/449,570, filed Jan. 23, 2017, both of which are incorporated by reference herein in their entireties for all purposes.

### FIELD

The subject matter described herein relates generally to systems, devices, and methods for using an applicator and a sensor control unit in an in vivo analyte monitoring system.

### BACKGROUND

The detection and/or monitoring of analyte levels, such as glucose, ketones, lactate, oxygen, hemoglobin A1C, or the like, can be vitally important to the health of an individual having diabetes. Patients suffering from diabetes mellitus can experience complications including loss of consciousness, cardiovascular disease, retinopathy, neuropathy, and nephropathy. Diabetics are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device may be worn on the body of an individual who requires analyte monitoring. To increase comfort and convenience for the individual, the sensor control device may have a small form-factor, and can be assembled and applied by the individual with a sensor applicator. The application process includes inserting a sensor, such as a dermal sensor that senses a user's analyte level in a bodily fluid located in the dermal layer of the human body, using an applicator or insertion mechanism, such that the sensor comes into contact with a bodily fluid. The sensor control device may also be configured to transmit analyte data to another device, from which the individual or her health care provider (“HCP”) can review the data and make therapy decisions.

While current sensors can be convenient for users, they are also susceptible to malfunctions due to improper insertion. These malfunctions can be caused by user error, lack of proper training, poor user coordination, overly complicated procedures, and other issues. This can be particularly true for analyte monitoring systems having dermal sensors, which are typically of smaller scale relative to sensors used to measure an analyte level in an interstitial fluid (“ISF”), and which are inserted using sharps (also known as “introducers” or “needles”) that are shorter than those used for ISF sensors. Some prior art systems, for example, may rely too much on the precision assembly and deployment of a sensor control device and an applicator by the individual user. Other prior art systems may utilize sharp insertion and retraction mechanisms that are susceptible to premature withdrawal before the sensor can be properly implanted. In addition, with respect to dermal sensors, some prior art systems may utilize sharps that are not optimally configured to create an insertion path in the dermal layer without creating trauma to surrounding tissue. These challenges and others described herein can lead to improperly inserted or damaged sensors, and consequently, a failure to properly monitor the patient's analyte level.

Thus, a need exists for more reliable sensor insertion devices, systems and methods, particularly for use in conjunction with dermal sensors, that are easy to use by the patient and less prone to error.

### SUMMARY

Provided herein are example embodiments of systems, devices and methods for the assembly and use of an applicator and a sensor control device of an in vivo analyte monitoring system, and in particular, where dermal sensors are utilized. An applicator can be provided to the user in a sterile package with an electronics housing of the sensor control device contained therein. A structure separate from the applicator, such as a container, can also be provided to the user as a sterile package with a sensor module and a sharp module contained therein. The user can couple the sensor module to the electronics housing, and can couple the sharp to the applicator with an assembly process that involves the insertion of the applicator into the container in a specified manner. After assembly, the applicator can be used to position the sensor control device on a human body with a sensor in contact with the wearer's bodily fluid (e.g., dermal fluid). The embodiments provided herein are improvements to prevent or reduce the likelihood that a sensor is improperly inserted or damaged Other improvements and advantages are provided as well. The various configurations of these devices are described in detail by way of the embodiments which are only examples.

Other systems, devices, methods, features and advantages of the subject matter described herein will be or will become apparent to one with skill in the art upon examination of the following figures and detailed description. It is intended that all such additional systems, devices, methods, features, and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of a sensor applicator, reader device, monitoring system, network, and remote system.

FIG. 2A is a block diagram depicting an example embodiment of a reader device.

FIGS. 2B and 2C are block diagrams depicting example embodiments of sensor control devices.

FIG. 3A is a proximal perspective view depicting an example embodiment of a user preparing a tray for an assembly.

FIG. 3B is a side view depicting an example embodiment of a user preparing an applicator device for an assembly.

FIG. 3C is a proximal perspective view depicting an example embodiment of a user inserting an applicator device into a tray during an assembly.

FIG. 3D is a proximal perspective view depicting an example embodiment of a user removing an applicator device from a tray during an assembly.

FIG. 3E is a proximal perspective view depicting an example embodiment of a patient applying a sensor using an applicator device.

FIG. 3F is a proximal perspective view depicting an example embodiment of a patient with an applied sensor and a used applicator device.

FIG. 4A is a side view depicting an example embodiment of an applicator device coupled with a cap.

FIG. 4B is a side perspective view depicting an example embodiment of an applicator device and cap decoupled.

FIG. 4C is a perspective view depicting an example embodiment of a distal end of an applicator device and electronics housing.

FIG. 5 is a proximal perspective view depicting an example embodiment of a tray with sterilization lid coupled.

FIG. 6A is a proximal perspective cutaway view depicting an example embodiment of a tray with sensor delivery components.

FIG. 6B is a proximal perspective view depicting sensor delivery components.

FIG. 7A is side view depicting an example embodiment of a housing.

FIG. 7B is a perspective view depicting an example embodiment of a distal end of a housing.

FIG. 7C is a side cross-sectional view depicting an example embodiment of a housing.

FIGS. 7D and 7E are side cross-sectional views depicting a locking rib portion of an example embodiment of a housing with a portion of a sheath.

FIGS. 7F and 7G are side cross-sectional views depicting a locking rib portion of another example embodiment of a housing and a portion of a sheath.

FIG. 7H is a side cross-sectional view depicting a locking rib portion of another example embodiment of a housing and a portion of a sheath.

FIG. 7I is a side cross-sectional view depicting a locking rib portion of another example embodiment of a housing and a portion of a sheath.

FIG. 8A is a side view depicting an example embodiment of a sheath.

FIG. 8B is a perspective view depicting an example embodiment of a proximal end of a sheath.

FIG. 8C is a close-up perspective view depicting an example embodiment of a distal side of a detent snap of a sheath.

FIG. 8D is a side view depicting an example embodiment of features of a sheath.

FIG. 8E is an end view of an example embodiment of a proximal end of a sheath.

FIGS. 8F to 8H are perspective views depicting another example embodiment of a sheath in various stages of assembly with other applicator components.

FIG. 9A is a proximal perspective view depicting an example embodiment of a sensor electronics carrier.

FIG. 9B is a distal perspective view depicting an example embodiment of a sensor electronics carrier.

FIG. 9C is a distal perspective view depicting another example embodiment of a sensor electronics carrier.

FIG. 9D is a side cross-sectional view depicting another example embodiment of a sensor electronics carrier along with housing and sheath.

FIG. 9E is a close-up side cross-sectional view depicting another example embodiment of a sensor electronics carrier along with housing.

FIG. 10A is a proximal perspective view of an example embodiment of a sharp carrier.

FIG. 10B is a side cross-section depicting an example embodiment of a sharp carrier.

FIG. 10C is a side cross-sectional view depicting another example embodiment of a sharp carrier assembly within an applicator.

FIG. 10D is a side cross-sectional view with a call-out depicting another example embodiment of a sharp carrier assembly along with a portion of a sensor electronics carrier.

FIG. 10E is a side cross-sectional view depicting another example embodiment of a sharp carrier assembly along with a portion of a sensor electronics carrier.

FIG. 10F is a side cross-sectional view depicting another example embodiment of a sharp carrier assembly and sheath within an applicator.

FIG. 11A is a perspective view depicting an example embodiment of a sharp module.

FIG. 11B is a perspective view of another example embodiment of a sharp module.

FIGS. 11C and 11D are schematic views depicting the sharp module of FIG. 11B.

FIGS. 11E and 11F are a side schematic view and a top-down schematic view, respectively, of the sharp module of FIG. 11B, as assembled with a sensor module.

FIG. 11G is a perspective view of another example embodiment of a sharp module.

FIG. 11H is a side schematic view depicting the sharp module of FIG. 11G.

FIGS. 11I and 11J are a side cross-sectional view and a side view, respectively, of the sharp module of FIG. 11G, as assembled with a sensor module.

FIGS. 12A to 12D are side cross-sectional views depicting an example embodiment of an applicator device during various stages of deployment.

FIGS. 13A to 13D are side cross-sectional views depicting another example embodiment of an applicator device during various stages of deployment.

FIGS. 14A to 14C are side cross-sectional views depicting another example embodiment of an applicator device during various stages of deployment. FIGS. 14A-1, 14A-2, 14B-1, 14B-2, 14C-1 and 14C-2 are call-out views of the example embodiment depicted in FIGS. 14A to 14C.

FIGS. 15A and 15B are side cross-sectional views depicting another example embodiment of an applicator device during various stages of deployment.

FIGS. 16A to 16C are side cross-sectional views depicting another example embodiment of an applicator device during various stages of deployment.

FIG. 17 is a side cross-sectional view depicting another example embodiment of an applicator device. FIG. 17-1 is a call-out that enlarges a portion of FIG. 17.

FIG. 18 is a partial cross-sectional view depicting another example embodiment of an applicator device.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include systems, devices, and methods for the use of dermal sensor insertion applicators for use with in vivo analyte monitoring systems. Accordingly, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices are disclosed, and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These sensor control device embodiments can be used and can be capable of use to implement those steps performed by a sensor control device from any and all of the methods described herein.

As mentioned, a number of embodiments of systems, devices, and methods are described herein that provide for the improved assembly and use of dermal sensor insertion devices for use with in vivo analyte monitoring systems. In particular, several embodiments of the present disclosure are designed to improve the method of sensor insertion with respect to in vivo analyte monitoring systems and, in particular, to prevent the premature retraction of an insertion sharp during a sensor insertion process. Some embodiments, for example, include a dermal sensor insertion mechanism with an increased firing velocity and a delayed sharp retraction. In other embodiments, the sharp retraction mechanism can be motion-actuated such that the sharp is not retracted until the user pulls the applicator away from the skin. Consequently, these embodiments can reduce the likelihood of prematurely withdrawing an insertion sharp during a sensor insertion process; decrease the likelihood of improper sensor insertion; and decrease the likelihood of damaging a sensor during the sensor insertion process, to name a few advantages. Several embodiments of the present disclosure also provide for improved insertion sharp modules to account for the small scale of dermal sensors and the relatively shallow insertion path present in a subject's dermal layer. In addition, several embodiments of the present disclosure are designed to prevent undesirable axial and/or rotational movement of applicator components during sensor insertion. Accordingly, these embodiments can reduce the likelihood of instability of a positioned dermal sensor, irritation at the insertion site, damage to surrounding tissue, and breakage of capillary blood vessels resulting in fouling of the dermal fluid with blood, to name a few advantages. In addition, to mitigate inaccurate sensor readings which can be caused by trauma at the insertion site, several embodiments of the present disclosure can reduce the end-depth penetration of the needle relative to the sensor tip during insertion.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g, automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Example Embodiment of In Vivo Analyte Monitoring System

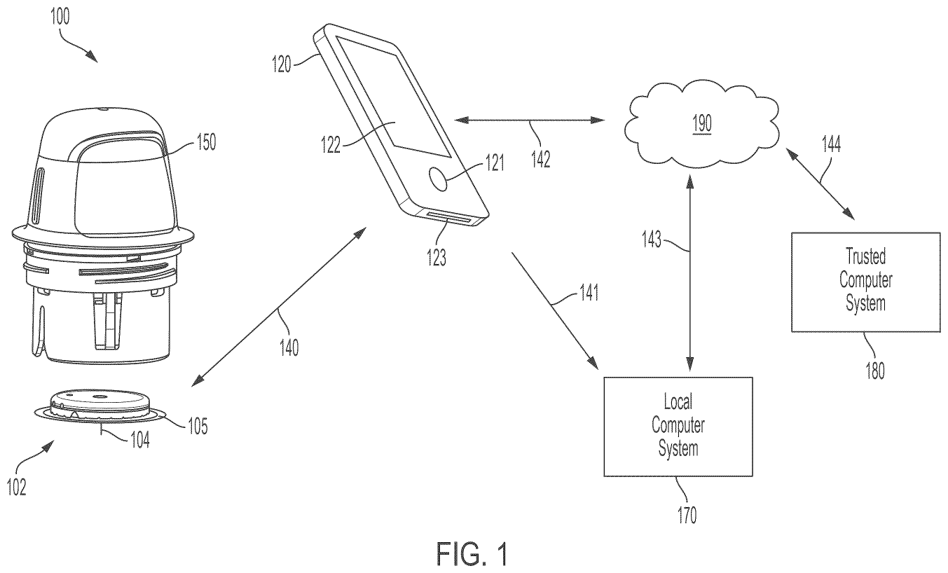

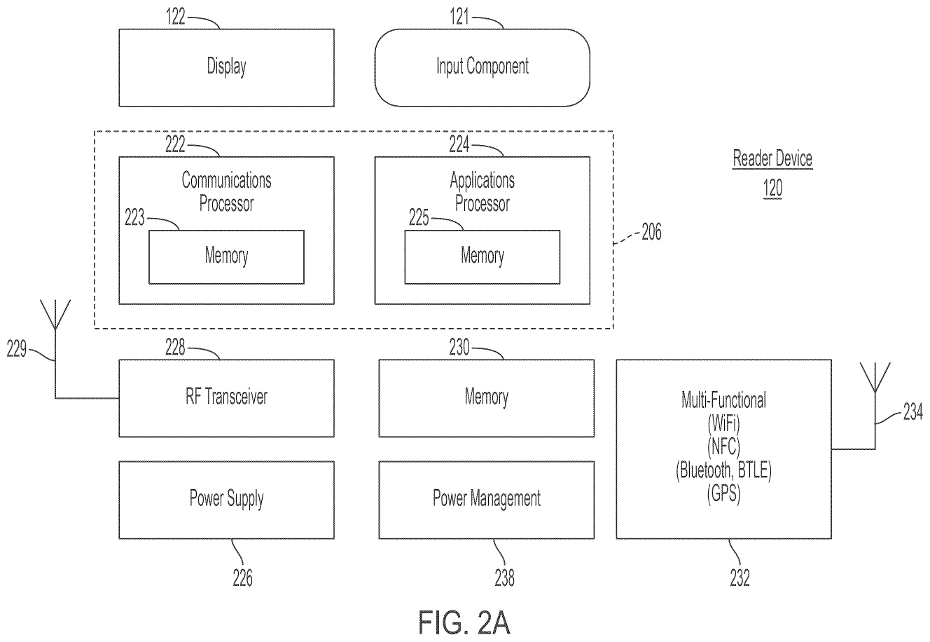

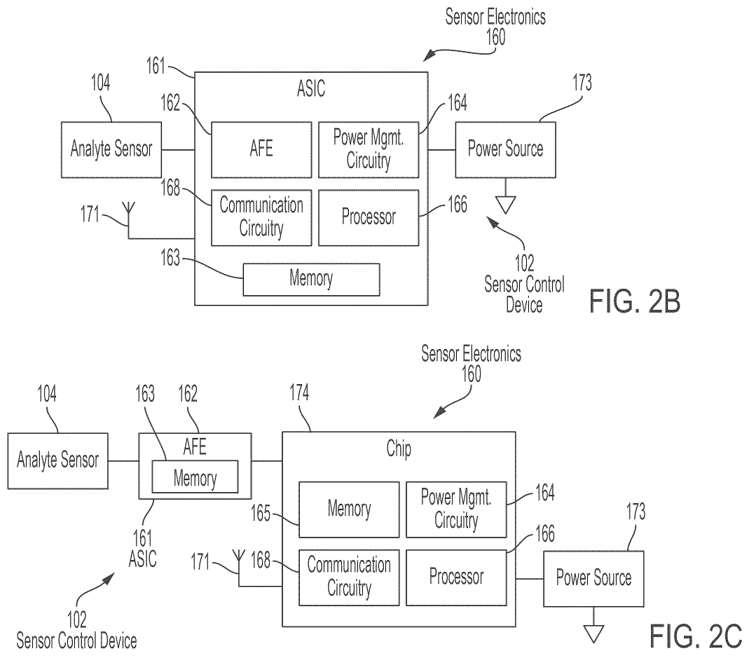

FIG. 1 is a conceptual diagram depicting an example embodiment of an analyte monitoring system 100 that includes a sensor applicator 150, a sensor control device 102, and a reader device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can monitor applications installed in memory on reader device 120 using screen 122 and input 121 and the device battery can be recharged using power port 123. More detail about reader device 120 is set forth with respect to FIG. 2A below. Reader device 120 can communicate with local computer system 170 via a communication path 141 using a wired or wireless technique. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how reader device 120 can communicate via a communications path 142 with network 190, by wired or wireless technique as described previously. Network 190 can be any of a number of networks, such as private networks and public networks, local area or wide area networks, and so forth. A trusted computer system 180 can include a server and can provide authentication services and secured data storage and can communicate via communications path 144 with network 190 by wired or wireless technique.

### Example Embodiment of Reader Device

FIG. 2A is a block diagram depicting an example embodiment of a reader device configured as a smartphone. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further included can be a multi-functional transceiver 232 which can communicate over Wi-Fi, NFC, Bluetooth, BTLE, and GPS with an antenna 234. As understood by one of skill in the art, these components are electrically and communicatively coupled in a manner to make a functional device.

### Example Embodiments of Sensor Control Device

FIGS. 2B and 2C are block diagrams depicting example embodiments of sensor control device 102 having analyte sensor 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 173, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 includes memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Example Embodiment of Assembly Process for Sensor Control Device

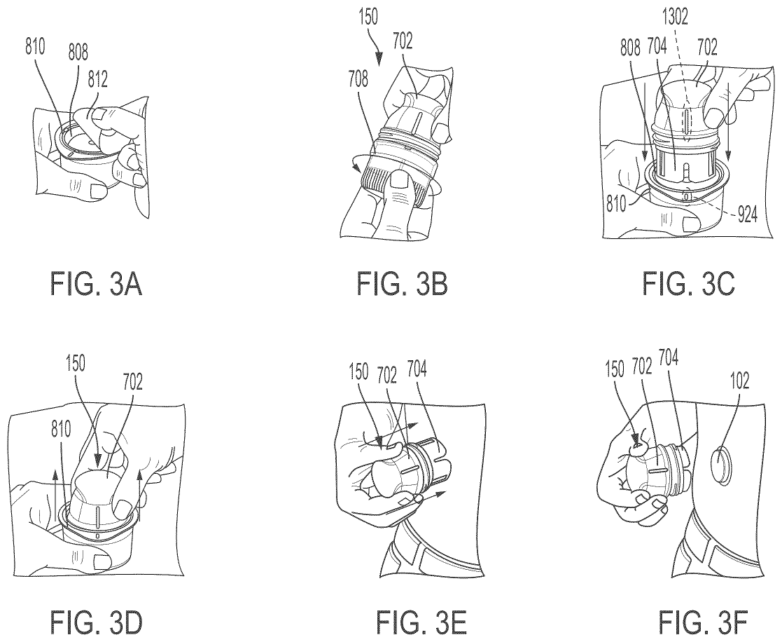

The components of sensor control device 102 can be acquired by a user in multiple packages requiring final assembly by the user before delivery to an appropriate user location. FIGS. 3A-3D depict an example embodiment of an assembly process for sensor control device 102 by a user, including preparation of separate components before coupling the components in order to ready the sensor for delivery. FIGS. 3E-3F depict an example embodiment of delivery of sensor control device 102 to an appropriate user location by selecting the appropriate delivery location and applying device 102 to the location.

FIG. 3A is a proximal perspective view depicting an example embodiment of a user preparing a container 810, configured here as a tray (although other packages can be used), for an assembly process. The user can accomplish this preparation by removing lid 812 from tray 810 to expose platform 808, for instance by peeling a non-adhered portion of lid 812 away from tray 810 such that adhered portions of lid 812 are removed. Removal of lid 812 can be appropriate in various embodiments so long as platform 808 is adequately exposed within tray 810. Lid 812 can then be placed aside.

FIG. 3B is a side view depicting an example embodiment of a user preparing an applicator device 150 for assembly. Applicator device 150 can be provided in a sterile package sealed by a cap 708. Preparation of applicator device 150 can include uncoupling housing 702 from cap 708 to expose sheath 704 (FIG. 3C). This can be accomplished by unscrewing (or otherwise uncoupling) cap 708 from housing 702. Cap 708 can then be placed aside.

FIG. 3C is a proximal perspective view depicting an example embodiment of a user inserting an applicator device 150 into a tray 810 during an assembly. Initially, the user can insert sheath 704 into platform 808 inside tray 810 after aligning housing orienting feature 1302 (or slot or recess) and tray orienting feature 924 (an abutment or detent). Inserting sheath 704 into platform 808 temporarily unlocks sheath 704 relative to housing 702 and also temporarily unlocks platform 808 relative to tray 810. At this stage, removal of applicator device 150 from tray 810 will result in the same state prior to initial insertion of applicator device 150 into tray 810 (i.e., the process can be reversed or aborted at this point and then repeated without consequence).

Sheath 704 can maintain position within platform 808 with respect to housing 702 while housing 702 is distally advanced, coupling with platform 808 to distally advance platform 808 with respect to tray 810. This step unlocks and collapses platform 808 within tray 810. Sheath 704 can contact and disengage locking features (not shown) within tray 810 that unlock sheath 704 with respect to housing 702 and prevent sheath 704 from moving (relatively) while housing 702 continues to distally advance platform 808. At the end of advancement of housing 702 and platform 808, sheath 704 is permanently unlocked relative to housing 702. A sharp and sensor (not shown) within tray 810 can be coupled with an electronics housing (not shown) within housing 702 at the end of the distal advancement of housing 702. Operation and interaction of the applicator device 150 and tray 810 are further described below.

FIG. 3D is a proximal perspective view depicting an example embodiment of a user removing an applicator device 150 from a tray 810 during an assembly. A user can remove applicator 150 from tray 810 by proximally advancing housing 702 with respect to tray 810 or other motions having the same end effect of uncoupling applicator 150 and tray 810. The applicator device 150 is removed with sensor control device 102 (not shown) fully assembled (sharp, sensor, electronics) therein and positioned for delivery.

FIG. 3E is a proximal perspective view depicting an example embodiment of a patient applying sensor control device 102 using applicator device 150 to a target area of skin, for instance, on an abdomen or other appropriate location. Advancing housing 702 distally collapses sheath 704 within housing 702 and applies the sensor to the target location such that an adhesive layer on the bottom side of sensor control device 102 adheres to the skin. The sharp is automatically retracted when housing 702 is fully advanced, while the sensor (not shown) is left in position to measure analyte levels.

FIG. 3F is a proximal perspective view depicting an example embodiment of a patient with sensor control device 102 in an applied position. The user can then remove applicator 150 from the application site.

System 100, described with respect to FIGS. 3A-3F and elsewhere herein, can provide a reduced or eliminated chance of accidental breakage, permanent deformation, or incorrect assembly of applicator components compared to prior art systems. Since applicator housing 702 directly engages platform 808 while sheath 704 unlocks, rather than indirect engagement via sheath 704, relative angularity between sheath 704 and housing 702 will not result in breakage or permanent deformation of the arms or other components. The potential for relatively high forces (such as in conventional devices) during assembly will be reduced, which in turn reduces the chance of unsuccessful user assembly.

### Example Embodiment of Sensor Applicator Device

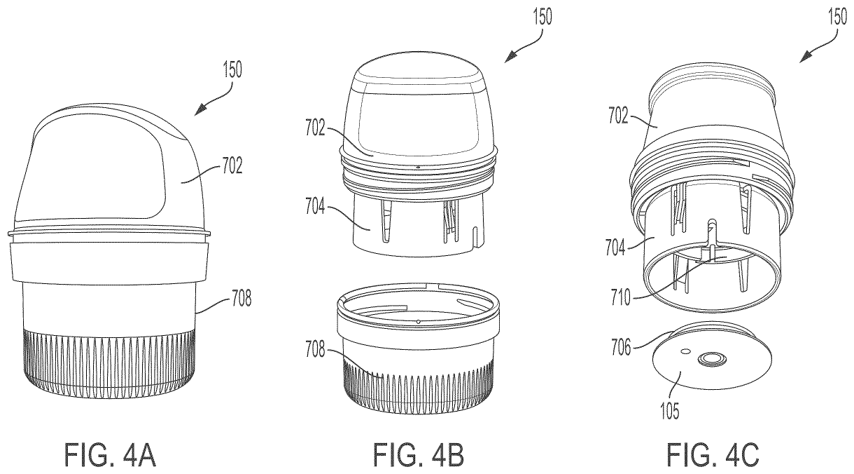

FIG. 4A is a side view depicting an example embodiment of an applicator device 150 coupled with screw cap 708. This is an example of how applicator 150 is shipped to and received by a user, prior to assembly by the user with a sensor. FIG. 4B is a side perspective view depicting applicator 150 and cap 708 after being decoupled. FIG. 4C is a perspective view depicting an example embodiment of a distal end of an applicator device 150 with electronics housing 706 and adhesive patch 105 removed from the position they would have retained within sensor electronics carrier 710 of sheath 704, when cap 708 is in place.

### Example Embodiment of Tray and Sensor Module Assembly

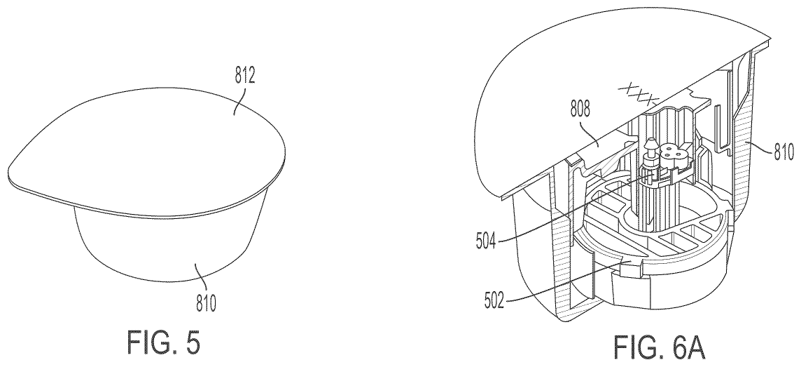

FIG. 5 is a proximal perspective view depicting an example embodiment of a tray 810 with sterilization lid 812 removably coupled thereto, which may be representative of how the package is shipped to and received by a user prior to assembly.

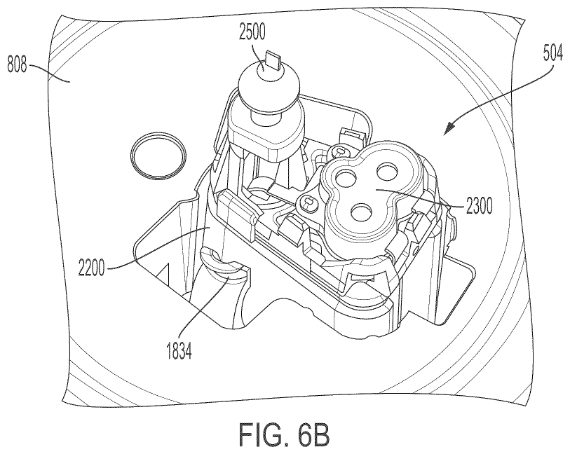

FIG. 6A is a proximal perspective cutaway view depicting sensor delivery components within tray 810. Platform 808 is slidably coupled within tray 810. Desiccant 502 is stationary with respect to tray 810. Sensor module 504 is mounted within tray 810.

FIG. 6B is a proximal perspective view depicting sensor module 504 in greater detail. Here, retention arm extensions 1834 of platform 808 releasably secure sensor module 504 in position. Module 2200 is coupled with connector 2300, sharp module 2500 and sensor (not shown) such that during assembly they can be removed together as sensor module 504

### Example Embodiment of Applicator Housing

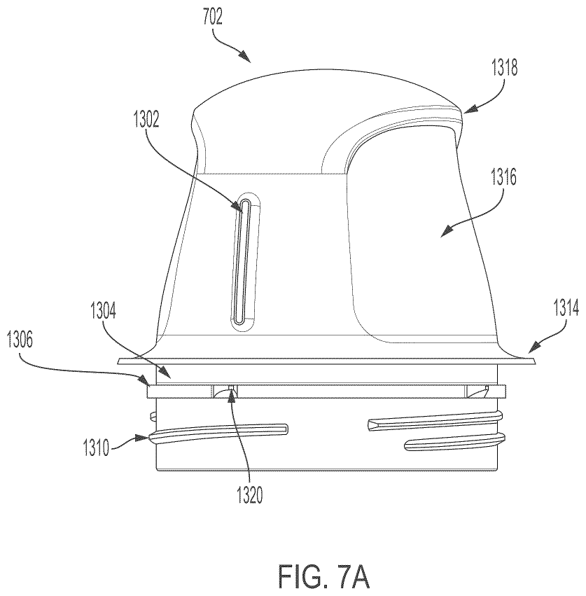

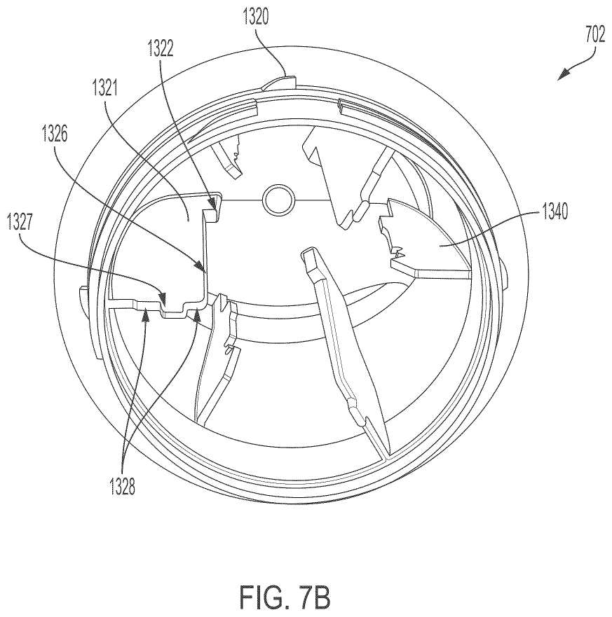

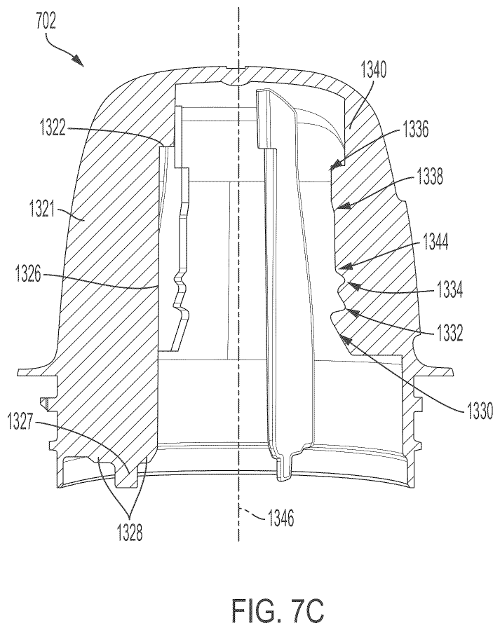

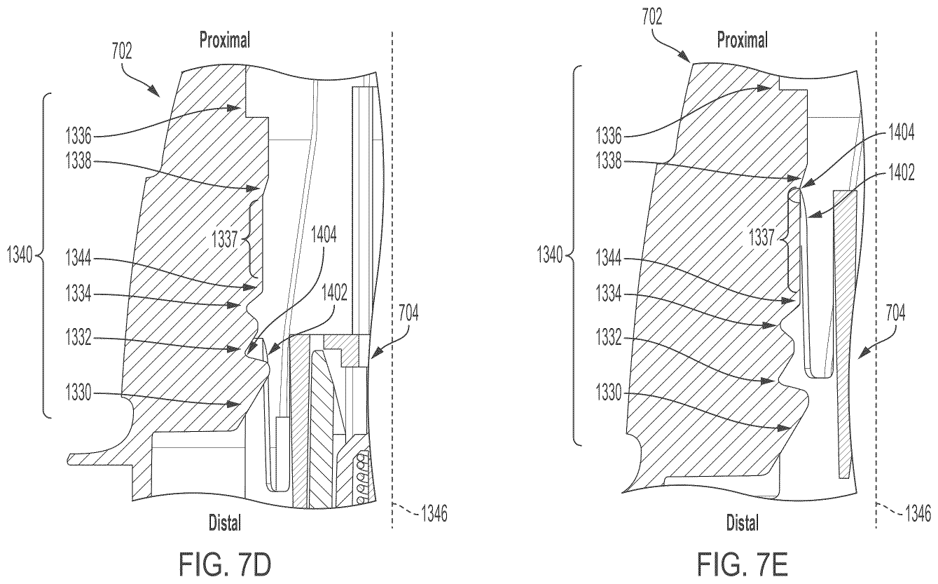

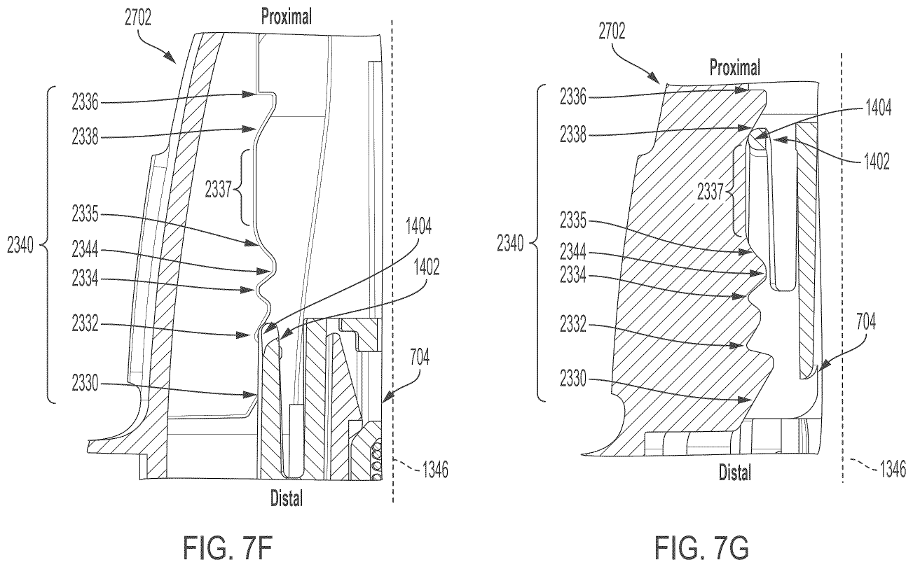

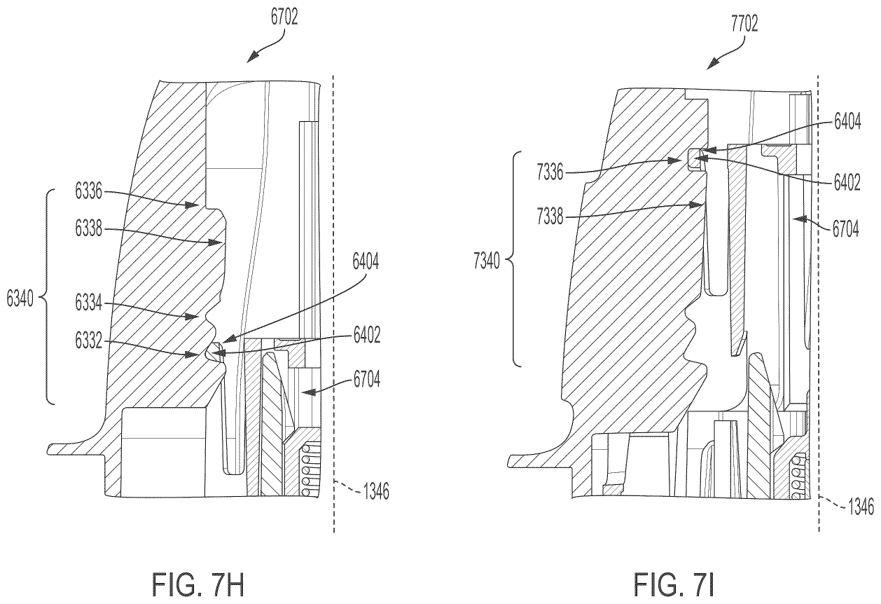

FIG. 7A is side view depicting an example embodiment of the applicator housing 702 that can include an internal cavity with support structures for applicator function. A user can push housing 702 in a distal direction to activate the applicator assembly process and then also to cause delivery of sensor control device 102, after which the cavity of housing 702 can act as a receptacle for a sharp. In the example embodiment, various features are shown including housing orienting feature 1302 for orienting the device during assembly and use. Tamper ring groove 1304 can be a recess located around an outer circumference of housing 702, distal to a tamper ring protector 1314 and proximal to a tamper ring retainer 1306. Tamper ring groove 1304 can retain a tamper ring so users can identify whether the device has been tampered with or otherwise used. Housing threads 1310 can secure housing 702 to complimentary threads on cap 708 (FIGS. 4A and 4B) by aligning with complimentary cap threads and rotating in a clockwise or counterclockwise direction. A side grip zone 1316 of housing 702 can provide an exterior surface location where a user can grip housing 702 in order to use it. Grip overhang 1318 is a slightly raised ridge with respect to side grip zone 1316 which can aid in ease of removal of housing 702 from cap 708. A shark tooth 1320 can be a raised section with a flat side located on a clockwise edge to shear off a tamper ring (not shown), and hold tamper ring in place after a user has unscrewed cap 708 and housing 702. In the example embodiment four shark teeth 1320 are used, although more or less can be used as desired.

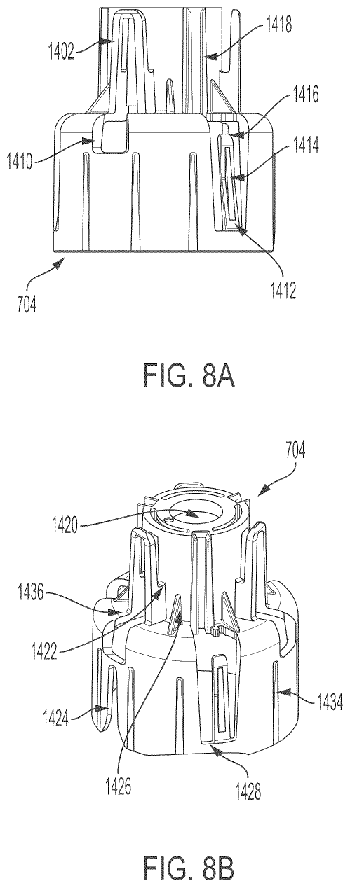

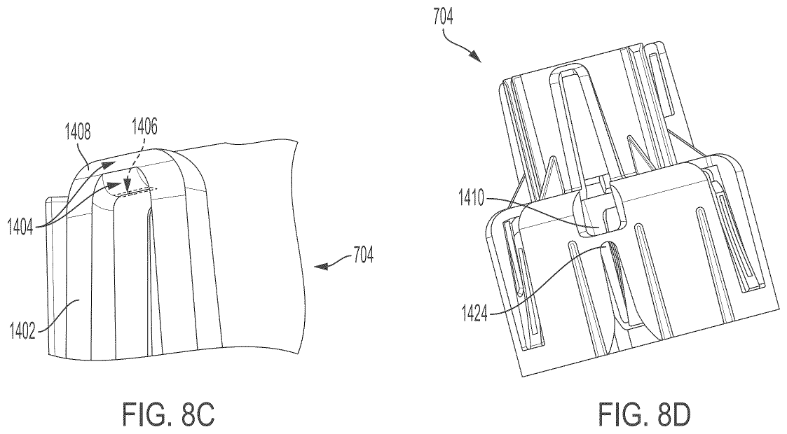

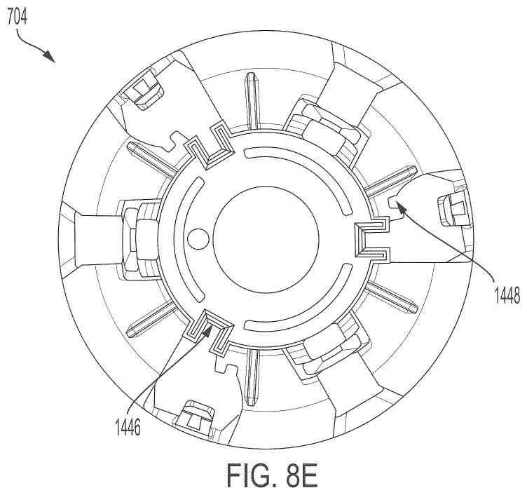

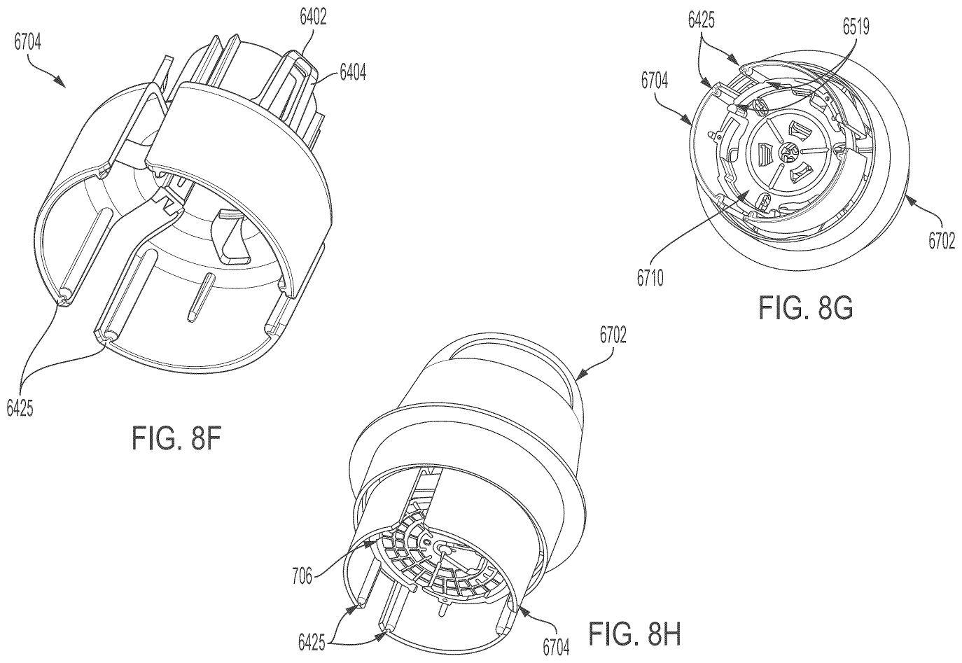

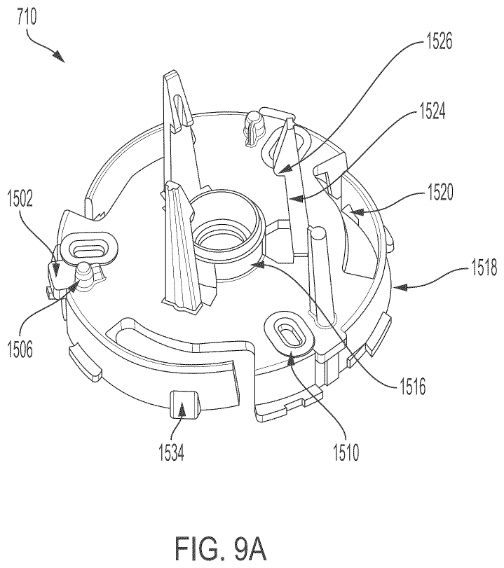

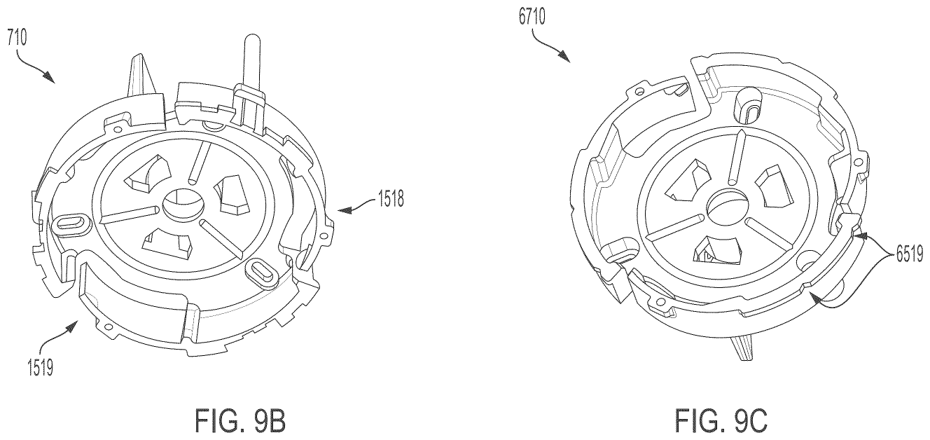

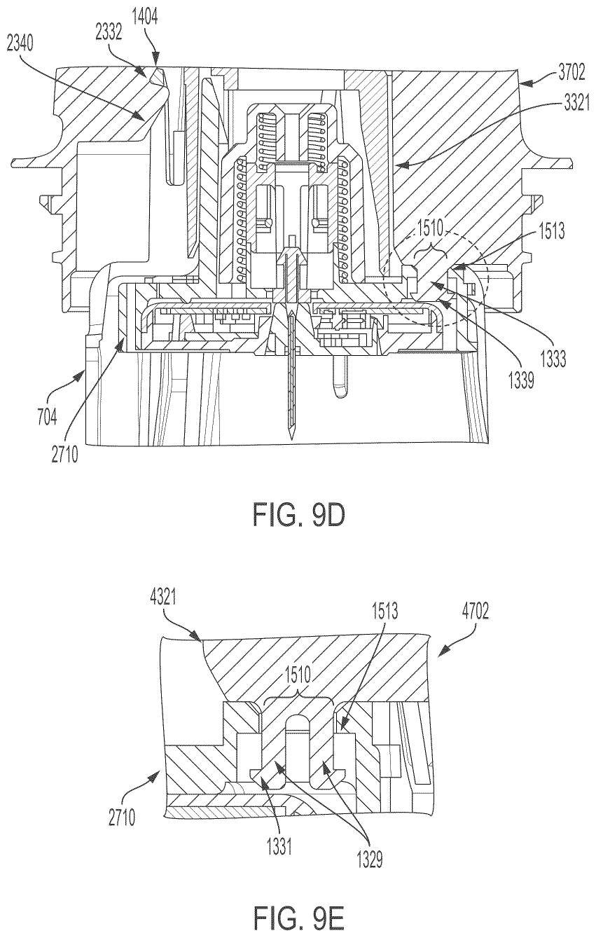

FIG. 7B is a perspective view depicting a distal end of housing 702. Here, three housing guide structures (or “guide ribs”) 1321 are located at 120 degree angles with respect to each other and at 60 degree angles with respect to locking structures (or “locking ribs”) 1340, of which there are also three at 120 degree angles with respect to each other. Other angular orientations, either symmetric or asymmetric, can be used, as well as any number of one or more structures 1321 and 1340. Here, each structure 1321 and 1340 is configured as a planar rib, although other shapes can be used. Each guide rib 1321 includes a guide edge (also called a “sheath guide rail”) 1326 that can pass along a surface of sheath 704 (e.g., guide rail 1418 described with respect to FIG. 8A). An insertion hard stop 1322 can be a flat, distally facing surface of housing guide rib 1321 located near a proximal end of housing guide rib 1321. Insertion hard stop 1322 provides a surface for a sensor electronics carrier travel limiter face 1420 of a sheath 704 (FIG. 8B) to abut during use, preventing sensor electronics carrier travel limiter face 1420 from moving any further in a proximal direction. A carrier interface post 1327 passes through an aperture 1510 (FIG. 9A) of sensor electronics carrier 710 during an assembly. A sensor electronics carrier interface 1328 can be a rounded, distally facing surface of housing guide ribs 1321 which interfaces with sensor electronics carrier 710.

FIG. 7C is a side cross-section depicting an example embodiment of a housing. In the example embodiment, side cross-sectional profiles of housing guide rib 1321 and locking rib 1340 are shown Locking rib 1340 includes sheath snap lead-in feature 1330 near a distal end of locking rib 1340 which flares outward from central axis 1346 of housing 702 distally. Each sheath snap lead-in feature 1330 causes detent snap round 1404 of detent snap 1402 of sheath 704 as shown in FIG. 8C to bend inward toward central axis 1346 as sheath 704 moves towards the proximal end of housing 702. Once past a distal point of sheath snap lead-in feature 1330, detent snap 1402 of sheath 704 is locked into place in locked groove 1332. As such, detent snap 1402 cannot be easily moved in a distal direction due to a surface with a near perpendicular plane to central axis 1346, shown as detent snap flat 1406 in FIG. 8C.

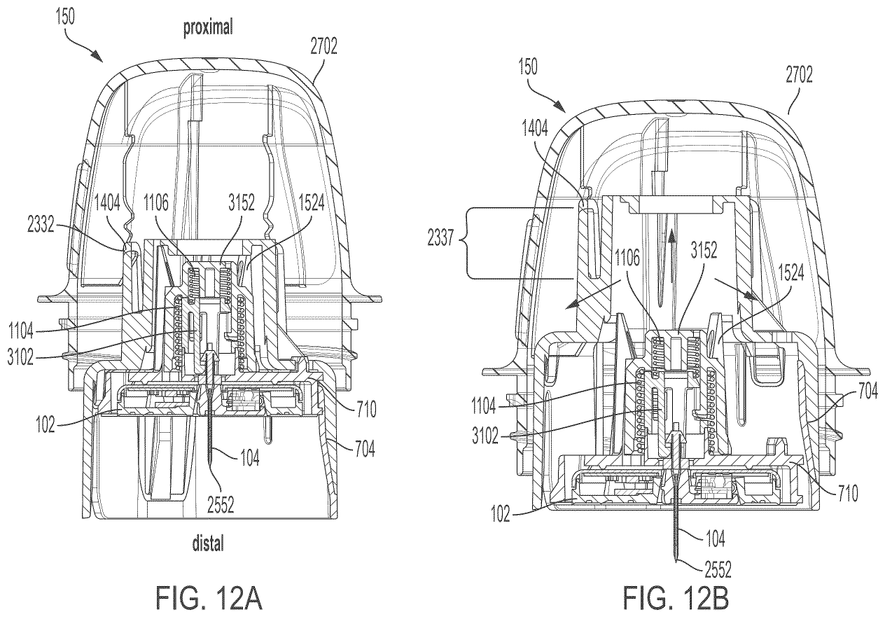

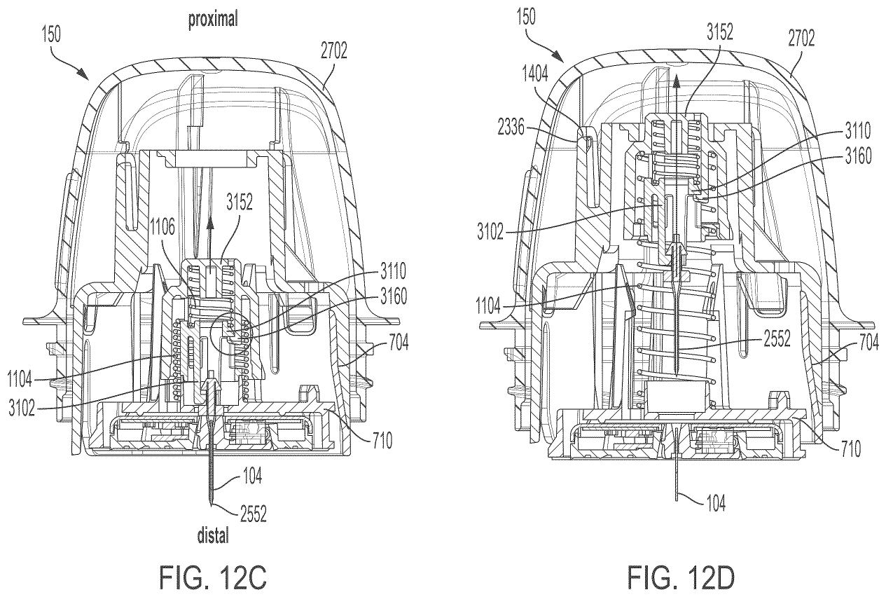

As housing 702 moves further in a proximal direction toward the skin surface, and as sheath 704 advances toward the distal end of housing 702, detent snaps 1402 shift into the unlocked grooves 1334, and applicator 150 is in an “armed” position, ready for use. When the user further applies force to the proximal end of housing 702, while sheath 704 is pressed against the skin, detent snap 1402 passes over firing detent 1344. This begins a firing sequence (as described, for example, with respect to FIGS. 12A-12D) due to release of stored energy in the deflected detent snaps 1402, which travel in a proximal direction relative to the skin surface, toward sheath stopping ramp 1338 which is slightly flared outward with respect to central axis 1346 and slows sheath 704 movement during the firing sequence. The next groove encountered by detent snap 1402 after unlocked groove 1334 is final lockout groove 1336 which detent snap 1402 enters at the end of the stroke or pushing sequence performed by the user. Final lockout recess 1336 can be a proximally-facing surface that is perpendicular to central axis 1346 which, after detent snap 1402 passes, engages a detent snap flat 1406 and prevents reuse of the device by securely holding sheath 704 in place with respect to housing 702. Insertion hard stop 1322 of housing guide rib 1321 prevents sheath 704 from advancing proximally with respect to housing 702 by engaging sensor electronics carrier travel limiter face 1420.

FIGS. 7D and 7E are close-up side views of an example embodiment of locking rib 1340 of applicator housing 702, as detent snap 1402 of sheath 704 moves toward the proximal end of housing 702. FIG. 7D shows sheath 704 in a “locked” state, in which detent round 1404 of detent snap 1402 has already passed over sheath snap lead-in feature 1330 and is positioned in locked groove 1332 of locking rib 1340. As force is applied to the proximal end of housing 702, detent round 1404 is advanced proximally into unlocked groove 1334, placing applicator 150 into an “armed” position. When force is further applied to the proximal end of housing 702, applicator 150 is “fired,” as detent round 1404 is advanced proximally from the unlocked groove 1334 and passes over firing detent 1344. Thereafter, sheath 704 is further advanced proximally such that detent round 1404 is slidably advanced over firing surface 1337, as shown in FIG. 7E. In this embodiment, firing surface 1337 is substantially parallel to central axis 1346. As sheath 704 continues to advance proximally, detent round 1404 reaches sheath stopping ramp 1338 which slows the movement of sheath 704. Upon detent round 1404 reaching final lockout recess 1336, detent snap flat 1406 (not shown) is engaged and securely holds sheath 704 in place with respect to housing 702.

FIGS. 7F and 7G are close-up side views of an alternative embodiment of locking rib 2340 that is designed to improve the firing velocity of the sharp from the sensor applicator. Here, locking rib 2340 includes an inward detent ramp 2335 to reduce friction between sheath 704 and housing 2702 during firing. Locking rib 2340 also includes a sheath stopping ramp 2338 at the proximal end of firing surface 2337. In FIG. 7F, sheath 704 is initially shown in a “locked” state, in which detent round 1404 of detent snap 1402 has already passed over sheath snap lead-in feature 2330, and is positioned in locked groove 2332. As force is applied to the proximal end of housing 2702, detent round 1404 is advanced into unlocked groove 2334, placing applicator 150 into the “armed” position. When force is further applied to the proximal end of housing 2702, applicator 150 is “fired,” as detent round 1404 passes over firing detent 2344.

As shown in FIG. 7G, detent round 1404 then advances toward the proximal end of housing 2702 in a “free flight” state, in which detent round 1404 passes over inward detent ramp 2335. While advancing proximally in the “free flight” state, detent round 1404 can be in non-continuous, or have no contact with, inward detent ramp 2335 and firing surface 2337. In this regard, detent round 1404 can be easily and quickly advanced, as there is little to no frictional force between detent round 1404 and inward detent ramp 2335 and firing surface 2337, and as such, improves upon the firing velocity of the sharp from the applicator. Sheath stopping ramp 2338, which is positioned proximally further along the locking rib 2340 relative to the embodiment shown in FIGS. 7D and 7E, provides an edge portion to frictionally engage the detent round 1404 and slow the movement of sheath 704. The sheath stopping ramp 2338 can have a sloped shape and provide for increasing frictional contact as the detent round 1404 advances in a proximal direction. Finally, upon detent round 1404 reaching final lockout recess 2336, detent snap flat 1406 (not shown) is engaged and securely holds sheath 704 in place with respect to housing 2702. Lockout recess 2336 prevents detent round 1404 and sheath 704 from backwards, or distal movement. This embodiment reflects a higher firing velocity relative to the embodiment depicted in FIGS. 7D and 7E, which also assists in prevention of a premature withdrawal of sharp.

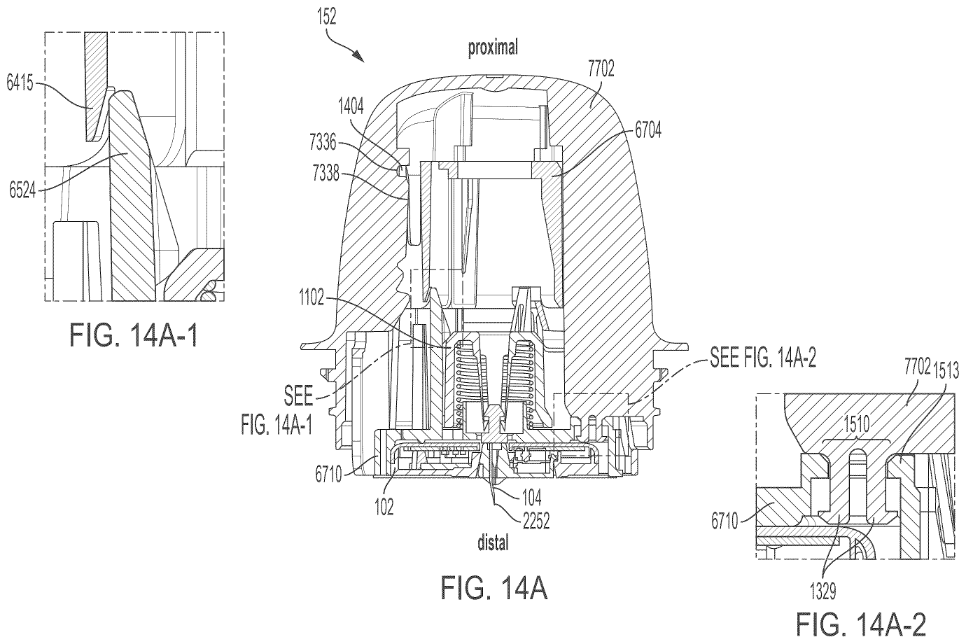

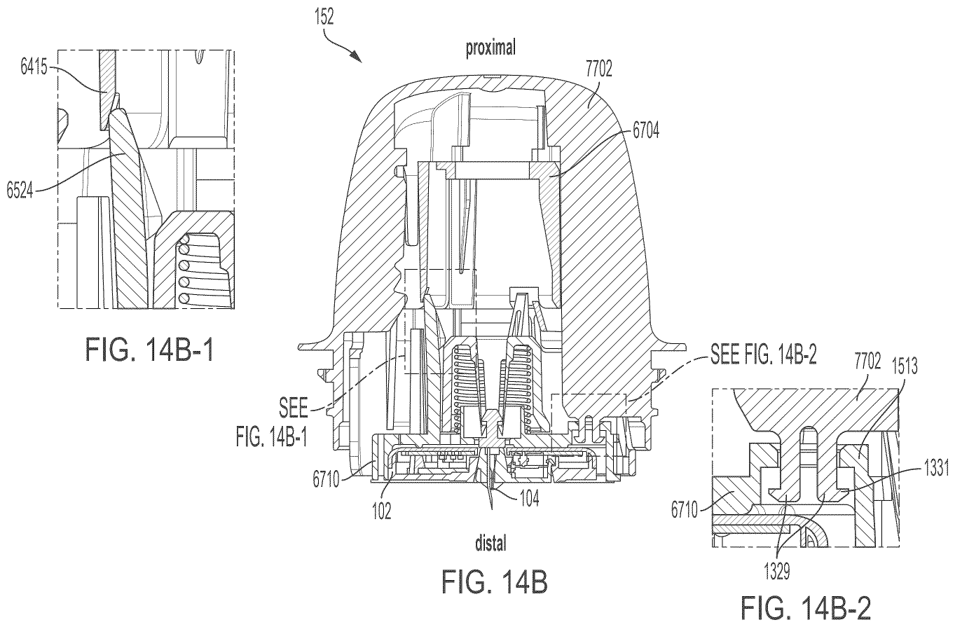

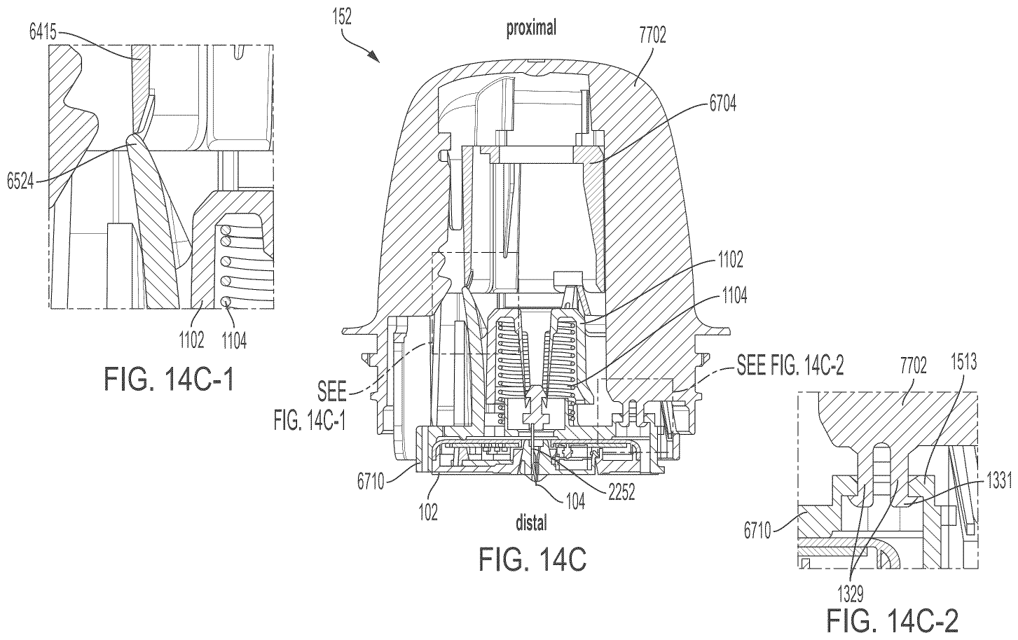

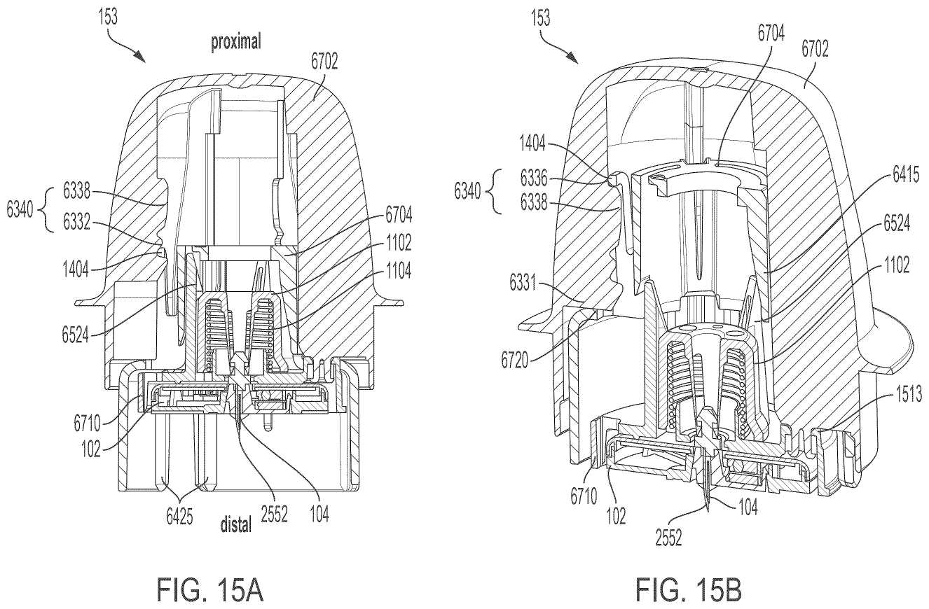

FIG. 7H is a close-up side view of an alternative embodiment of locking rib 6340 designed to maintain a downward force on sheath 6704 during firing which, in turn, can prevent sheath 6704 from unwanted movement during the sensor insertion process. Here, sheath 6704 is shown in a “locked” state, in which detent round 6404 of detent snap 6402 is positioned in locked groove 6332. As force is applied to the proximal end of housing 6702, detent round 6404 is advanced into unlocked groove 6334, placing applicator in the “armed” position. When force is further applied to the proximal end of housing 6702, applicator is “fired,” and detent round 6404 advances over sloped firing surface 6338 toward the proximal end of housing 6702. Sloped firing surface 6338 can be angled toward central axis 1346 such that the resulting downward force upon sheath 6704 increases as detent round 6404 advances in a proximal direction. In the depicted embodiment, detent round 6404 is in continuous contact with sloped firing surface 6338. Lockout recess 6336 prevents detent round 6404 and sheath 6704 from backwards, or distal movement. This embodiment reflects a slower firing velocity relative to the previously described embodiments, and can be used, for example, with the motion-actuated sharp retraction process that is described with respect to FIGS. 14A-14C and 15A-15B.

FIG. 71 is a close-up side view of still another alternative embodiment of locking rib 7340, also designed to maintain a downward force on sheath 6704 during firing which, in turn, can prevent sheath 6704 from unwanted movement during a sensor insertion process. Here, sheath 6704 is shown in a “fired” state, in which detent round 6404 of detent snap 6402 is positioned in a two-way lockout recess 7336. Upon detent round 6404 advancing into two-way lockout recess 7336, sheath 6704 can be prevented from further movement in either a proximal or distal direction. This can reduce unwanted movement of sheath 6704 during the sensor insertion process. Furthermore, in some embodiments, as described with respect to FIGS. 14A-14C and 15A-15B, two-way lockout recess 7336 can provide for the immobilization of sheath 6704 during a motion-actuated sharp retraction process. As can be seen in FIG. 71, sloped firing surface 7338 is angled toward central axis 1346 such that a resulting downward force upon sheath 6704 increases as detent round 6404 advances in a proximal direction. In the depicted embodiment, detent round 6404 is in continuous contact with sloped firing surface 7338. This embodiment reflects a slower firing velocity and can be used, for example, with the motion-actuated sharp retraction process that is described with respect to FIGS. 14A-14C and 15A-15B.

### Example Embodiment of Applicator Sheath

FIGS. 8A and 8B are a side view and perspective view, respectively, depicting an example embodiment of sheath 704. In this example embodiment, sheath 704 can stage sensor control device 102 above a user's skin surface prior to application. Sheath 704 can also contain features that help retain a sharp in a position for proper application of a sensor, determine the force required for sensor application, and guide sheath 704 relative to housing 702 during application. Detent snaps 1402 are near a proximal end of sheath 704, described further with respect to FIG. 8C below. Sheath 704 can have a generally cylindrical cross section with a first radius in a proximal section (closer to top of figure) that is shorter than a second radius in a distal section (closer to bottom of figure). Also shown are a plurality of detent clearances 1410, three in the example embodiment. Sheath 704 can include one or more detent clearances 1410, each of which can be a cutout with room for sheath snap lead-in feature 1330 to pass distally into until a distal surface of locking rib 1340 contacts a proximal surface of detent clearance 1410.

Guide rails 1418 are disposed between sensor electronics carrier traveler limiter face 1420 at a proximal end of sheath 704 and a cutout around lock arms 1412. Each guide rail 1418 can be a channel between two ridges where the guide edge 1326 of housing guide rib 1321 can slide distally with respect to sheath 704.

Lock arms 1412 are disposed near a distal end of sheath 704 and can include an attached distal end and a free proximal end, which can include lock arm interface 1416. Lock arms 1412 can lock sensor electronics carrier 710 to sheath 704 when lock arm interface 1416 of lock arms 1412 engage lock interface 1502 of sensor electronics carrier 710. Lock arm strengthening ribs 1414 can be disposed near a central location of each lock arm 1412 and can act as a strengthening point for an otherwise weak point of each lock arm 1412 to prevent lock arm 1412 from bending excessively or breaking.

Detent snap stiffening features 1422 can be located along the distal section of detent snaps 1402 and can provide reinforcement to detent snaps 1402. Alignment notch 1424 can be a cutout near the distal end of sheath 704, which provides an opening for user alignment with sheath orientation feature of platform 808. Stiffening ribs 1426 can include buttresses, that are triangularly shaped here, which provide support for detent base 1436. Housing guide rail clearance 1428 can be a cutout for a distal surface of housing guide rib 1321 to slide during use. Tilt reducing ribs 1434 are also located in a distal region of sheath 704.

FIG. 8C is a close-up perspective view depicting an example embodiment of detent snap 1402 of sheath 704. Detent snap 1402 can include a detent snap bridge 1408 located near or at its proximal end. Detent snap 1402 can also include a detent snap flat 1406 on a distal side of detent snap bridge 1408. An outer surface of detent snap bridge 1408 can include detent snap rounds 1404 which are rounded surfaces that allow for easier movement of detent snap bridge 1408 across interior surfaces of housing 702 such as, for example, locking rib 1340.

FIG. 8D is a side view depicting an example embodiment of sheath 704. Here, alignment notch 1424 can be relatively close to detent clearance 1410. Detent clearance 1410 is in a relatively proximal location on distal portion of sheath 704.

FIG. 8E is an end view depicting an example embodiment of a proximal end of sheath 704. Here, a back wall for guide rails 1446 can provide a channel to slidably couple with housing guide rib 1321 of housing 702. Sheath rotation limiter 1448 can be notches which reduce or prevent rotation of the sheath 704.

FIGS. 8F-8H are perspective views of an alternative example embodiment of sheath 6704 in various stages of assembly with other components of the applicator. As shown in FIG. 8F, sheath 6704 can have many of the same features as sheath 704, previously described with respect to FIGS. 8A-8C. Sheath 6704, for example, can include one or more detent snaps 6404 having one or more detent rounds 6402 attached thereto Sheath 6704, however, can be shorter in overall length as compared to sheath 702. In addition, sheath 6704 can include one or more inner sheath ribs 6425 disposed on the inner surface of sheath 6704, and which protrude in an inward direction towards the central axis of sheath 6704.

Turning to FIG. 8G, sheath 6704 is shown in perspective view in a stage of assembly with applicator housing 6702 and sensor electronics carrier 6710. One or more inner sheath ribs 6425 of sheath 6704 can interface with one or more corresponding rib notches 6519 in sensor electronics carrier 6710. The fitted interface between corresponding ribs 6425 and notches 6519 can help maintain axial alignment of the sheath 6704 and sensor electronics carrier 6710 during the sensor insertion process. Furthermore, the interface between ribs 6425 and notches 6519 can reduce lateral and rotational movement between the applicator components, which can, in turn, reduce the chance of improper sensor insertion.

Turning to FIG. 8H, sheath 6704 is shown in perspective view in a stage of assembly with applicator housing 6702 and sensor electronics housing 706, which has been inserted into sensor electronics carrier 6710. Inner sheath ribs 6425 are also shown.

It should be noted that although six inner sheath ribs 6425 and six corresponding rib notches 6519 are depicted, any number of ribs and notches are fully within the scope of the present disclosure. Moreover, while ribs 6425 are depicted with a rounded surface edge, in other embodiments, ribs 6425 can have a rectangular or triangular shape, and rib notches 6519 can have a corresponding receiving shape for interfacing with ribs 6425. In addition, although ribs 6425 are depicted as being disposed on an inner circumferential surface of sheath 6704, ribs 6425 can also be disposed on any other surface of sheath 6704, or portion thereof, that comes into contact with sensor electronics carrier 6710.

### Example Embodiments of Sensor Electronics Carriers

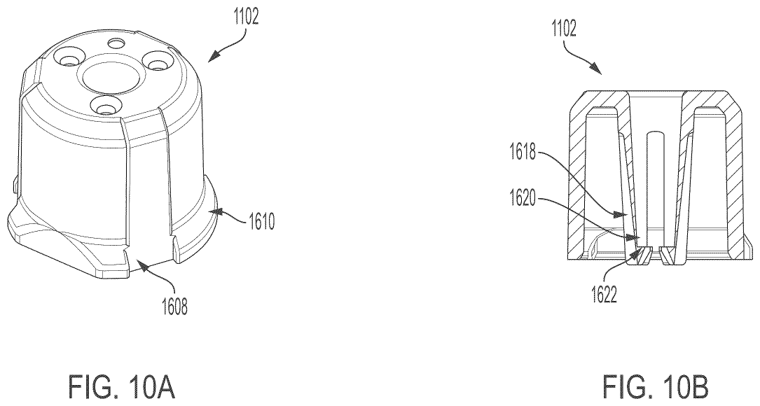

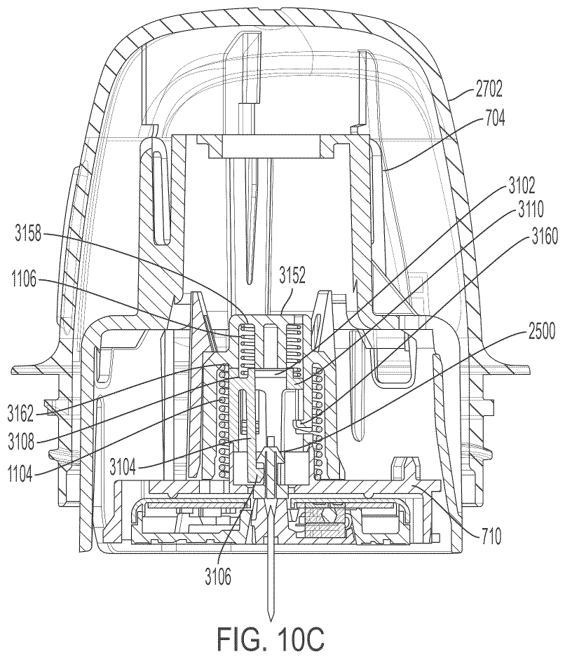

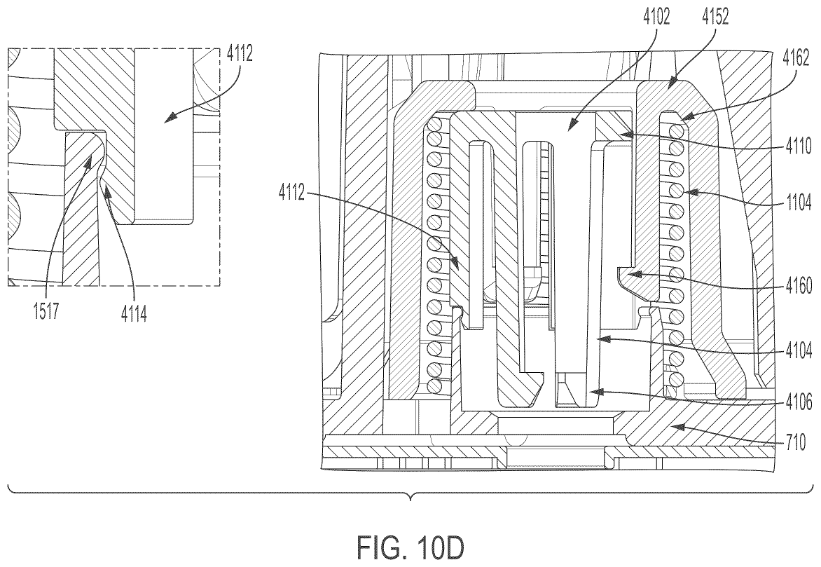

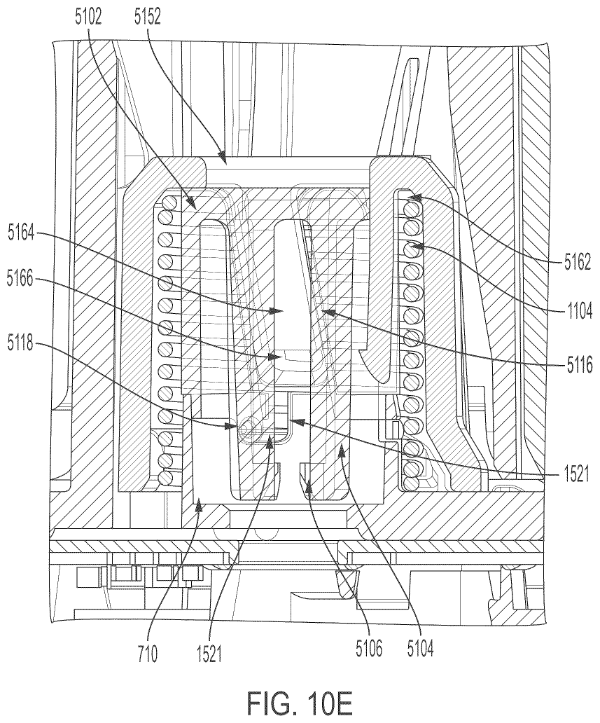

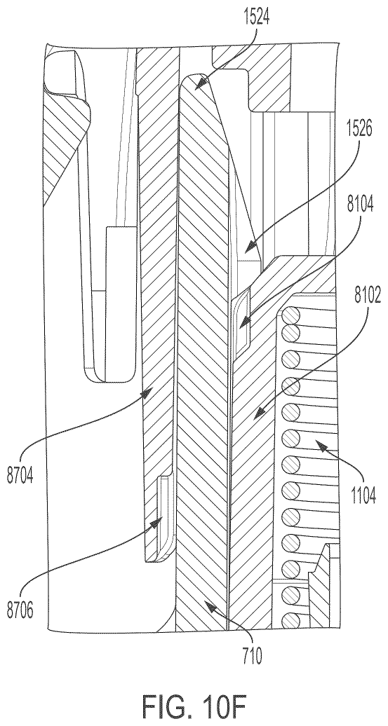

FIG. 9A is a proximal perspective view depicting an example embodiment of sensor electronics carrier 710 that can retain sensor electronics within applicator 150. It can also retain sharp carrier 1102 with sharp module 2500. In this example embodiment, sensor electronics carrier 710 generally has a hollow round flat cylindrical shape, and can include a sensor electronics retention feature 1520 and one or more deflectable sharp carrier lock arms 1524 (e.g., three) extending proximally from a proximal surface surrounding a centrally located spring alignment ridge 1516 for maintaining alignment of spring 1104. Each lock arm 1524 has a detent or retention feature 1526 located at or near its proximal end. Shock lock 1534 can be a tab located on an outer circumference of sensor electronics carrier 710 extending outward and can lock sensor electronics carrier 710 for added safety prior to firing Rotation limiter 1506 can be a proximally extending relatively short protrusion on a proximal surface of sensor electronics carrier 710 which limits rotation of carrier 710. Sharp carrier lock arms 1524 can interface with sharp carrier 1102 as described with reference to FIGS. 10A-10E below.

FIG. 9B is a distal perspective view of sensor electronics carrier 710. Here, one or more sensor electronics retention spring arms 1518 (e.g., three) are normally biased towards the position shown and include a detent 1519 that can pass over the distal surface of electronics housing 706 of device 102 when housed within recess or cavity 1521. In certain embodiments, after sensor control device 102 has been adhered to the skin with applicator 150, the user pulls applicator 150 in a proximal direction, i.e., away from the skin. The adhesive force retains sensor control device 102 on the skin and overcomes the lateral force applied by spring arms 1518. As a result, spring arms 1518 deflect radially outwardly and disengage detents 1519 from sensor control device 102 thereby releasing sensor control device 102 from applicator 150.

FIG. 9C is a perspective view of an alternative example embodiment of sensor electronics carrier 6710. As shown in FIG. 9C, sensor electronics carrier 6710 can have many of the same features as sensor electronics carrier 710, previously described with respect to FIGS. 9A-9B. In addition, sensor electronics carrier 6710 also includes one or more notch ribs 6519 disposed along an outer circumferential surface. As best seen in FIGS. 8F-8H, notch ribs 6519 are configured to interface with inner sheath ribs 6425 in order to maintain axial alignment of the sheath and sensor electronics carrier, and reduce lateral and rotational movement between applicator components during the sensor insertion process.

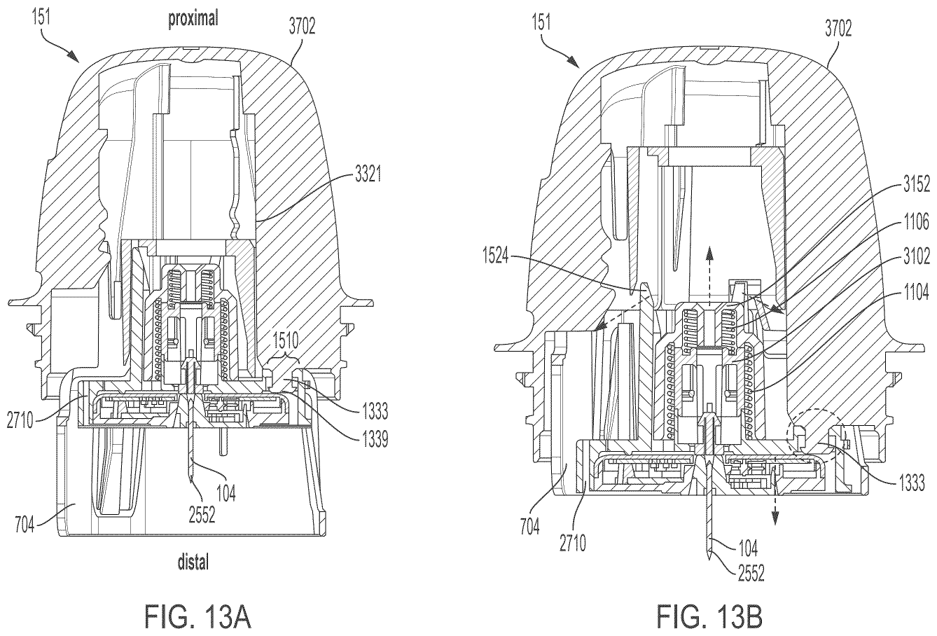

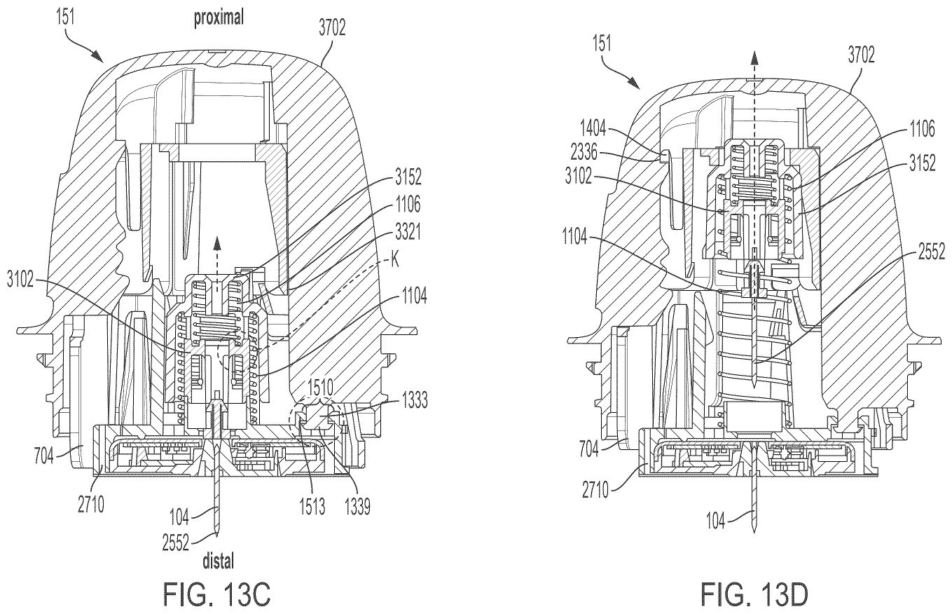

FIGS. 9D and 9E depict alternative embodiments of sensor electronics carriers for use with the insertion of dermal sensors. These embodiments include a retention mechanism to couple the applicator housing with the sensor electronics carrier, while also allowing for the sensor electronics carrier to advance a limited distance in a proximal-to-distal direction while the sharp is inserted into the skin. The retention mechanism can operate to further increase the velocity of sharp insertion during firing, while delaying the sharp retraction, as further described below and with respect to FIGS. 13A-13D. In other embodiments (e.g., as shown in FIGS. 14A-14C and 15A-15B), the retention mechanism can also provide for a displacement area between the sensor electronics carrier and sheath, through which a motion-actuated sharp retention mechanism can be initiated.

FIG. 9D is a side cross-sectional view of an alternative embodiment of sensor electronics carrier 2710, shown here with applicator housing 3702 and sheath 704. Here, applicator 150 is depicted in a “locked state,” in which detent round 1404 of sheath 704 is positioned in locked groove 2332 of locking rib 2340 of housing 3702. At a distal end of housing guide rib 3321 of housing 3702, a heat stake post 1333 is provided. Heat stake post 1333 can protrude in a distal direction through aperture 1510 of sensor electronics carrier 2710. Distal portion 1339 of heat stake post 1333 can be flared such that the distal portion is larger than aperture 1510 of sensor electronics carrier 2710, and prevents heat stake post 1333 from sliding out of aperture 1510 due to impedance of aperture ledge 1513. Heat stake post 1333 can have a length greater than the thickness of aperture ledge 1513, allowing for spaced movement between sensor electronics carrier 2710 and housing 3702 along a longitudinal axis through the center of heat stake post 1333 (as further depicted in FIGS. 13A-13D). As shown in FIG. 9D, when applicator 150 is depicted in the “locked state,” the proximal end (or base) of heat stake post 1333 is near to, or flush against, sensor electronics carrier 2710, aperture 1510 and aperture ledge 1513. During a firing sequence, sensor electronics carrier 2710 is displaced in a distal direction, creating a spaced relation between the proximal end (or base) of heat stake post 1333 and sensor electronics carrier 2710, aperture 1510 and aperture ledge 1513.

FIG. 9E is a side cross-sectional view of sensor electronics carrier 710 and an alternative embodiment of housing 4702. At a distal end of housing guide rib 4321 of housing 4702, one or more snap-in arms 1329 are provided. Snap-in arms 1329 can protrude in a distal direction through aperture 1510 of sensor electronics carrier 2710. A snap-in detent 1331 is provided at the end of each snap-in arm 1329. Snap-in detents 1331 can be flared such that the distal ends of snap-in arms 1329 are larger than the aperture 1510 of sensor electronics carrier 2710, and prevent snap-in arms 1329 from completely exiting out of aperture 1510 due to aperture ledge 1513. Snap-in arms 1329 can also have a length greater than the thickness of ledge 1513, allowing for spaced movement between sensor electronics carrier 2710 and housing 4702 along a longitudinal axis. The movement of the embodiments depicted in FIG. 9E during the “locked” and “firing” stages are similar to the movement of the embodiments shown in FIG. 9D, and further illustrated in FIGS. 12A-12D and 13A-13D. Additionally, the embodiments described with respect to FIGS. 9D and 9E can also be implemented with a motion-actuated sharp retraction mechanism, which is further described with respect to FIGS. 14A-14C and 15A-15B.

### Example Embodiments of Sharp Carriers

FIGS. 10A and 10B are a proximal perspective view and a side cross-sectional view, respectively, depicting an example embodiment of sharp carrier 1102. Sharp carrier 1102 can grasp and retain sharp module 2500 within applicator 150. It can also automatically retract as a result of one or more springs changing from a preloaded, compressed state to an expanded state during an insertion process, as described with respect to FIGS. 12A-12D and 13A-13D. Near a distal end of sharp carrier 1102 can be anti-rotation slots 1608 which prevent sharp carrier 1102 from rotating when located within a central area of sharp carrier lock arms 1524 (as shown in FIG. 9A). Anti-rotation slots 1608 can be located between sections of sharp carrier base chamfer 1610, which can ensure full retraction of sharp carrier 1102 through sheath 704 upon retraction of sharp carrier 1102 at the end of the deployment procedure.

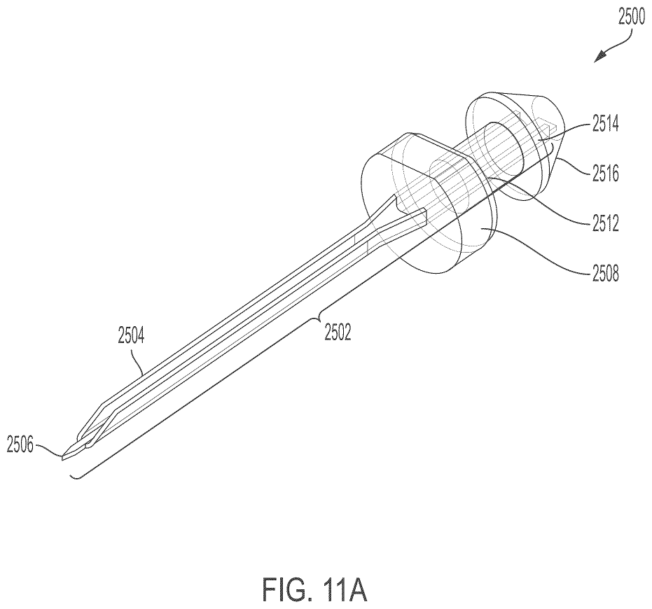

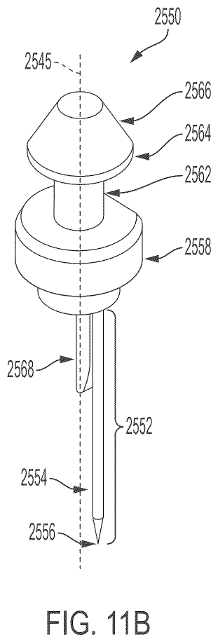

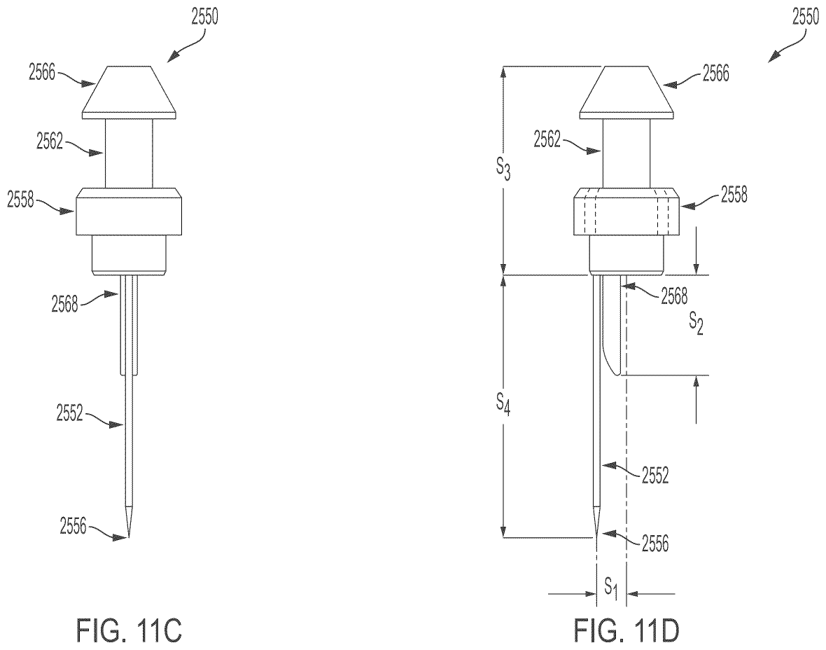

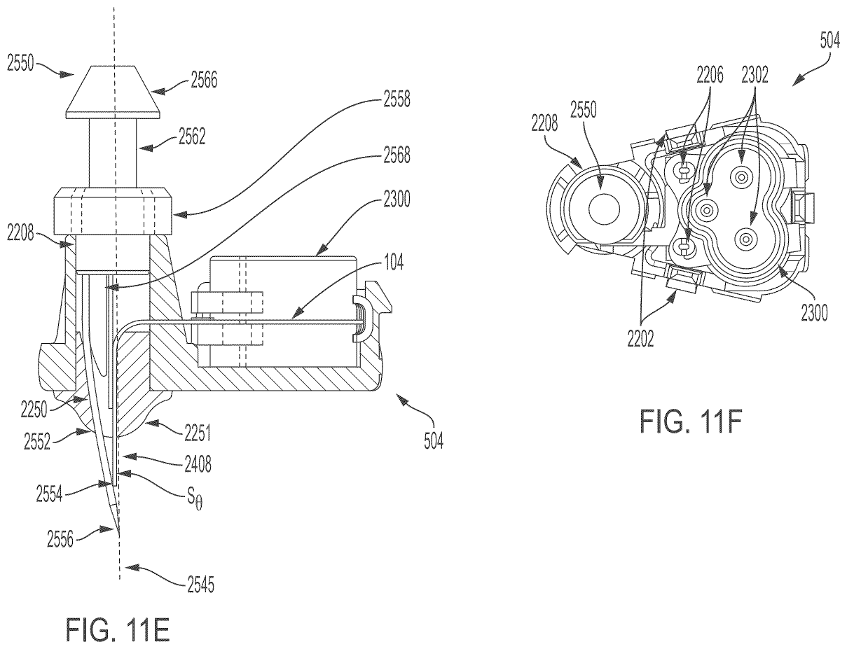

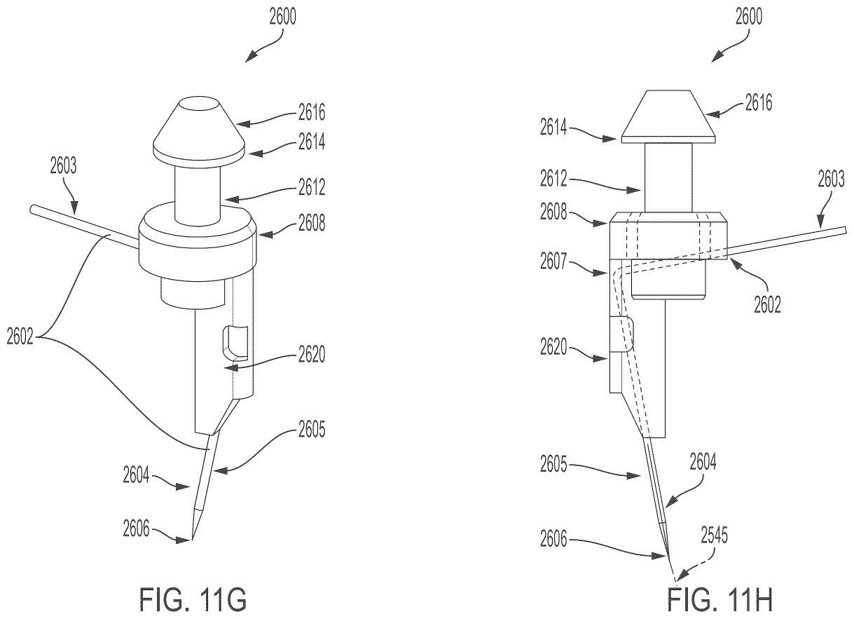

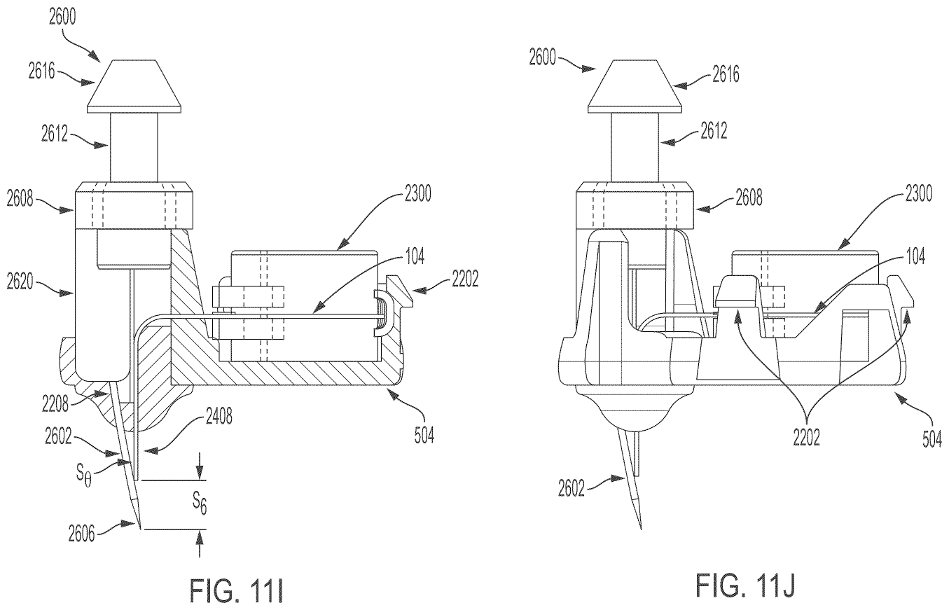

As shown in FIG. 10B, sharp retention arms 1618 can be located in an interior of sharp carrier 1102 about a central axis and can include a sharp retention clip 1620 at a distal end of each arm 1618. Sharp retention clip 1620 can have a proximal surface which can be nearly perpendicular to the central axis and can abut a distally facing surface of sharp hub 2516 (FIG. 11A).

FIGS. 10C to 10E depict alternative embodiments of sharp carrier assemblies, each of which consists of an inner sharp carrier and an outer sharp carrier. These embodiments provide for a delay, created by a separate retraction process for each sharp carrier, occurs during a firing sequence in which a dermal sensor is implanted into a subject's dermal layer prior to retraction of the sharp. The introduction of the delay can significantly reduce the likelihood of premature withdrawal of the sharp during the insertion process.

FIG. 10C is a side view of an alternative embodiment of a two-piece sharp carrier assembly consisting of inner sharp carrier 3102 and outer sharp carrier 3152, along with sensor electronics carrier 710, sheath 704, and housing 2702. Inner sharp carrier 3102 can include one or more sharp retention arms 3104 for retaining sharp module 2500. Sharp retention arms 3104 can further include a sharp retention clip 3106 located at a distal end of each arm 3104. Sharp retention clips 3106 can have a proximal surface that can be nearly perpendicular to a central axis and can abut a distally facing surface of sharp hub 2516, as shown in FIG. 11A. At a proximal end surface of inner sharp carrier 3102, a bottom inner spring retention channel 3108 is provided which can retain a distal end of inner spring 1106, which is shown in a preloaded and compressed state prior to retraction of the sharp carrier assembly. One or more inner carrier latches 3110 are also provided at or near a proximal end of inner sharp carrier 3102. Inner carrier latch 3110 can include a substantially flat surface that faces towards the distal end of applicator 150 and protrudes radially outward from a central longitudinal axis of inner sharp carrier 3102.

Still referring to FIG. 10C, outer sharp carrier 3152 can be external to and surround inner sharp carrier 3102. At a proximal end of outer sharp carrier 3152, a top inner spring retention channel 3158 is provided, which can retain a proximal end of inner spring 1106. Top inner spring retention channel 3158 of outer sharp carrier 3152 and bottom inner spring retention channel 3108 of inner sharp carrier 3102 each provide a surface to retain an end of inner spring 1106. Outer sharp carrier 3152 can also include an outer spring retention channel 3162 for retaining a proximal end of outer spring 1104, which is also shown in a preloaded and compressed state prior to the retraction of the sharp carrier assembly. As seen in FIG. 10C, outer spring 1104 is shown as having both a greater length and radius than inner spring 1106. However, springs 1104, 1106 can be of equal size and/or radius, or, in the alternative, inner spring 1106 may have a greater radius and/or length than outer spring 1104. In some embodiments, outer spring 1104 has an equal or greater stiffness than inner spring 1106.

Referring again to FIG. 10C, outer sharp carrier 3152 can also include one or more outer carrier latches 3160 Outer carrier latch 3160 can include a substantially flat surface that faces towards the proximal end of applicator 150 and protrudes radially inward towards a central longitudinal axis of outer sharp carrier 3152. The flat surface of outer carrier latch 3160 and the flat surface of inner carrier latch 3110 can be facing each other and aligned along a longitudinal axis extending from the proximal end to the distal end of applicator 150. As described in FIGS. 12A-12D and 13A-13D, inner carrier latch 3110 is positioned proximally to outer carrier latch 3160 in a spaced relation while applicator 150 is in a “locked” state. As sheath 704 is advanced in a proximal direction, applicator 150 is “fired,” and sharp carrier lock arms 1524 of sensor electronics carrier 710 are released into their biased outward position. Subsequently, forces generated by expansion of inner spring 1106 and outer spring 1104 cause outer sharp carrier 3152 to advance in a proximal direction. In addition, an opposing force generated by the expansion of inner spring 1106 causes the inner sharp carrier 3102 to remain in relatively the same position, thereby preventing premature retraction of sharp. Similarly, an opposing force generated by the expansion of outer spring 1104 causes sensor electronics carrier 710 to remain in relatively the same position (or displaced in a distal direction toward the skin surface). As outer sharp carrier 3152 further advances in a proximal direction, outer carrier latch 3160 engages inner carrier latch 3110. Proximal forces caused by the carrier latches 3160, 3110 cause inner sharp carrier 3102 to move in a proximal direction into applicator 150, thereby retracting the sharp (not shown).

FIG. 10D is a side view of another embodiment of a two-piece sharp carrier assembly, consisting of inner sharp carrier 4102 and outer sharp carrier 4152. Similar to the previous embodiment, inner sharp carrier 4102 can include one or more sharp retention arms 4104 with sharp retention clips 4106, and one or more inner carrier latches 4110 at or near a proximal end of inner sharp carrier 4102. Outer sharp carrier 4152 can also include a spring retention channel 4162 for retaining spring 1104, as well as outer carrier latch 4160 for interfacing with inner carrier latch 4110. These structures operate in a similar fashion as the embodiment described with respect to FIG. 10C.

Referring still to FIG. 10D, the two-piece sharp carrier assembly includes one spring 1104 (in contrast to the two springs depicted in FIG. 10C). In addition, an inner sharp carrier detent 4114 is provided at a distal portion of inner sharp carrier 4102 (as shown in call-out of FIG. 10D) for engaging with a carrier retention detent 1517 located on sensor electronics carrier 710. The engagement of inner sharp carrier detent 4114 with carrier retention detent 1517 causes inner sharp carrier 4102 and sensor electronics carrier 710 to remain locked in place while the sharp penetrates the skin surface during the insertion process. Inner sharp carrier detent 4114 can be disengaged from carrier retention detent 1517 during the “firing” of applicator 150. As sharp carrier lock arms 1524 of sensor electronics carrier 710 are released (as shown in FIGS. 12B and 13B), spring 1104 expands from its preloaded, compressed state. Subsequently, outer sharp carrier 4152 is advanced in a proximal direction while inner sharp carrier 4102 remains relatively in the same position, thereby preventing premature retraction of sharp. As outer sharp carrier 4152 continues to advance in a proximal direction, outer carrier latch 4160 engages inner carrier latch 4110, and a proximal force applied by the outer carrier latch 4160 to inner carrier latch 4110 causes inner sharp carrier detent 4114 to disengage from carrier retention detent 1517. Thereafter, outer carrier latch 4160 pulls inner sharp carrier 4102 in a proximal direction into applicator 150, thereby retracting the sharp (not shown).

With respect to FIG. 10D, those of skill in the art will understand that other retaining devices may be utilized in place of inner carrier detent arm 4112 and carrier retention detent 1517. For example, in alternative embodiments, snaps, hooks, ball locks, latches, pins or other like retaining devices can be utilized to maintain inner sharp carrier 4102 in a “locked” position with sensor electronics carrier 710 until a sufficient force from outer sharp carrier 4152 causes the retaining device to disengage, thereby allowing inner sharp carrier 4102 to advance in a proximal direction. In other alternative embodiments, a screw thread can be utilized between inner sharp carrier 4102 and sensor electronics carrier 710 to retain inner sharp carrier 4102 in position during the “firing” sequence of applicator 150 (as shown in FIGS. 12A-12D and 13A-13D). Subsequently, as outer sharp carrier 4152 continues to advance in a proximal direction, the proximal force of outer sharp carrier 4152 can cause inner sharp carrier 4102 to rotate and disengage itself from sensor electronics carrier 710. It should be understood that these exemplary retention devices and their equivalents are within the scope of the embodiments disclosed herein.

FIG. 10E is a side view of yet another embodiment of a two-piece sharp carrier, consisting of inner sharp carrier 5102 and outer sharp carrier 5152. Similar to the previous embodiment, inner sharp carrier 5102 can include one or more sharp retention arms 5104 with sharp retention clips 5106. Outer sharp carrier 5152 can include a spring retention channel 5162 for retaining spring 1104.

Referring still to FIG. 10E, outer sharp carrier 5152 can include one or more angled snap arms 5164 extending in an inward direction from a proximal top portion of outer sharp carrier 5152, such that each angled snap arm 5164 can slope in a downward direction towards a distal portion of inner sharp carrier 5102. Each angled snap arm 5164 can include at the distal end, a snap arm ledge 5166 which can consist of an end portion that provides a substantially flat surface facing in a proximal direction (i.e., akin to the outer carrier latch 4160 as described with respect to FIG. 10D). In addition, each distal end of the one or more angled snap arms 5164 can be in fitted contact with one or more angled key slots 5116 of inner sharp carrier 5102. Angled key slots 5116 can consist of cut-outs having a generally “tilted rectangular” shape, in an outer cylindrical surface of inner sharp carrier 5102, and extend circumferentially from a proximal end to a distal end of inner sharp carrier 5102.

Referring again to FIG. 10E, inner sharp carrier 5102 can also include one or more locking nubs 5118 on the outer cylindrical surface of a proximal portion of inner sharp carrier 5102. Locking nub 5118 can consist of a fixed spherical, hemispherical or otherwise rounded structure that protrudes in an outward direction, away from a central longitudinal axis of inner sharp carrier 5102, and can be in fitted contact with a carrier nub slot 1521 located on a distal portion of sensor electronics carrier 710. Carrier nub slot 1521 can consist of a cut-out in spring alignment ridge 1516 of sensor electronics carrier 710, in which the cut-out has an open end from which locking nub 5118 can slidably disengage upon rotation of inner sharp carrier 5102.

With reference to the embodiment shown in FIG. 10E, the relative movements of outer sharp carrier 5152, inner sharp carrier 5102 and spring 1104 during “firing” of applicator 150 will now be generally described. As sharp carrier lock arms 1524 of sensor electronics carrier 710 are released (shown in FIGS. 12B and 13B), spring 1104 expands from its preloaded, compressed state. Subsequently, outer sharp carrier 5152 is advanced in a proximal direction. Inner sharp carrier 5102 remains relatively in the same position due to locking nub 5118 being engaged in carrier nub slot 1521, thereby preventing premature retraction of sharp. As outer sharp carrier 5152 continues to advance in a proximal direction, the force exerted by angled snap arm 5164 upon angled key slot 5116 causes inner sharp carrier 5102 to rotate due to the angular orientation of angled key slot 5116. Due to the rotation of inner sharp carrier 5102, locking nub 5118 is slidably advanced toward the open end of carrier nub slot 1521 of sensor electronics carrier 710. When locking nub 5118 reaches the open end of carrier nub slot 1521, inner sharp carrier 5102 disengages from sensor electronics carrier 710. As outer sharp carrier 5152 further advances in a proximal direction, snap arm ledge 5166 engages the proximal end portion of angled key slot 5116, and begins to pull inner sharp carrier 5102 in a proximal direction into applicator 150, thereby retracting the sharp (not shown).

As shown in FIG. 10E, two angled snap arms 5164 and two angled key slots 5116 are depicted. It is to be understood, however, that any number of angled snap arms 5164 and/or angled key slots 5116 can be utilized. In addition, although carrier nub slot 1521 is shown in FIG. 10E as having an “L-shaped” cut-out, any number of cut-out shapes (e.g., “curve” or “linear slope”) having one open end from which locking nub 5118 can slidably disengage are suitable.

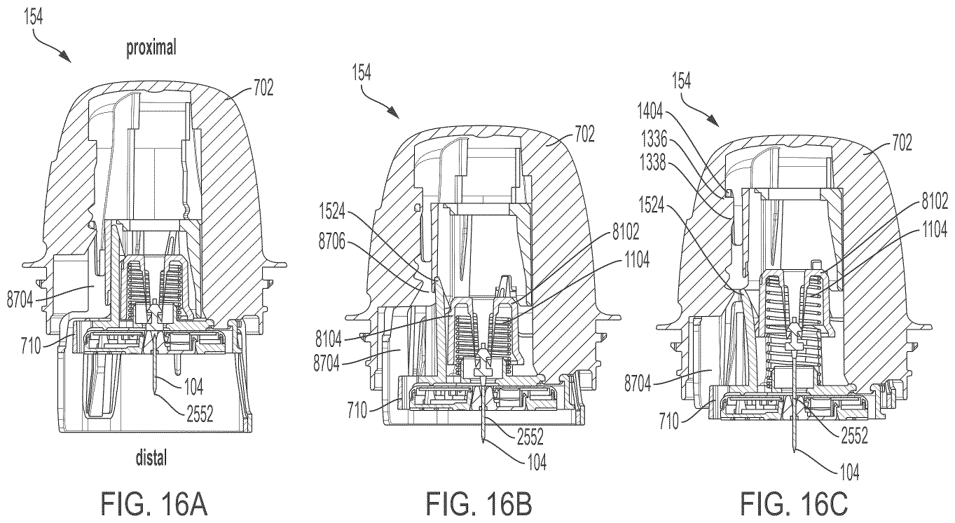

FIG. 10F is a close-up, side cross-sectional view depicting another example embodiment of a sharp carrier assembly 8102 and sheath 8704 within an applicator. According to one aspect of the embodiments, sharp carrier assembly 8102 can include a sharp carrier slot 8104 disposed on a surface of sharp carrier assembly 8102, and along the path upon which sharp carrier retention feature 1526 of the sensor electronics carrier 710 travels during retraction of the needle (not shown). Similarly, according to another aspect of the embodiments, sheath 8704 can include a sheath slot 8706 disposed on a surface of sheath 8704, and along the path upon which sharp carrier lock arm 1524 of sensor electronics carrier 710 travels during retraction of the needle. As further described below, with respect to FIGS. 16A-16C, sharp carrier slot 8104 and sheath slot 8706 can be configured to receive, respectively, sharp carrier lock retention feature 1526 and sharp carrier lock arm 1524 to allow for a dual-stage needle retraction process. In particular, according to some embodiments, as lock arms 1524 of sensor electronics carrier 710 are received into sharp carrier slot 8104 and sheath slot 8706, lock arms 1524 can partially deflect in an outward direction, which can cause the sharp carrier 8102 to move a limited distance in a proximal direction from the force of expansion of preloaded compression spring 1104 disposed in sharp carrier 8102. In this manner, the needle can be partially retracted, or maintained at a stationary position relative to the skin surface, such that further penetration into the subject's dermis or subcutaneous tissue by the needle can be prevented.

### Example Embodiments of Sharp Modules

FIG. 11A is a perspective view depicting an example embodiment of sharp module 2500 prior to assembly within sensor module 504 (FIG. 6B). Sharp 2502 can include a distal tip 2506 which can penetrate the skin while carrying sensor tail in a hollow or recess of sharp shaft 2504 to put the active surface of the sensor tail into contact with bodily fluid. A hub push cylinder 2508 can provide a surface for a sharp carrier to push during insertion. A hub small cylinder 2512 can provide a space for the extension of sharp hub contact faces 1622 (FIG. 10B). A hub snap pawl locating cylinder 2514 can provide a distal-facing surface of hub snap pawl 2516 for sharp hub contact faces 1622 to abut. A hub snap pawl 2516 can include a conical surface that opens clip 1620 during installation of sharp module 2500.

FIGS. 11B to 11H show example embodiments of sharp modules, in various stages of assembly, for use in the insertion of dermal analyte sensors. According to one aspect of the embodiments, angling the sensor and/or insertion sharp relative to a reference point can enable co-localization of the tip of the insertion needle and the tip of the sensor, and furthermore, can create a single contact point at the surface of the skin. As such, the sharp can create a leading edge at the surface of the skin to form an insertion path into the dermal layer for the sensor, as the sensor is inserted into a subject. In some embodiments, for example, the sharp and/or dermal sensor may be angled relative to a reference point (e.g., each other, surface of the skin, or the base of the applicator) for insertion, where the angle of the sharp differs from the angle of the sensor. For example, the reference point may be the skin surface to be breached for dermal insertion, or may be a reference or component of the sensor applicator set. In some embodiments, the sharp may be disposed at an angle relative to the sensor. For example, when designed so that that the sharp is angled relative to the sensor, the needle creates a leading edge for the sensor during operation of the applicator set. Furthermore, the needle design itself, and the positioning of the needle with respect to the sensor can be implemented in any desired configuration, including all of those configurations disclosed in U.S. Patent Publication No. 2014/0171771, which is incorporated by reference herein in its entirety for all purposes. Furthermore, although many of the example embodiments described with respect to FIGS. 11B to 11J make reference to dermal analyte sensors and dermal insertion, it will be understood by those of skill in the art that any of the embodiments can be dimensioned and configured for use with analyte sensors that can be positioned beyond the dermal space, such as into (or even fully through) subcutaneous tissue (e.g., 3 mm to 10 mm beneath the surface of the skin depending on the location of the skin on the body).

FIG. 11B is a perspective view depicting an example embodiment of a sharp module 2550 that can be used for the insertion of a dermal sensor. Sharp module 2550 is shown here prior to assembly with sensor module 504 (FIG. 6B), and can include components similar to those of the embodiment described with respect to FIG. 11A, including sharp 2552, sharp shaft 2554, sharp distal tip 2556, hub push cylinder 2558, hub small cylinder 2562, hub snap pawl 2566 and hub snap pawl locating cylinder 2564. Sharp 2552 can be positioned within sharp module 2550 at an off-center location relative to a longitudinal axis 2545 that extends through center of hub snap pawl 2566, hub small cylinder 2562 and hub push cylinder 2558. In addition, sharp module 2550 can include a sharp spacer 2568 that is parallel to and adjacent with a portion of sharp 2552. Sharp spacer 2568 can be positioned in between sensor 104 (not shown) and sharp 2552 along a proximal portion of sharp 2552, and can ensure that sensor 104 and sharp 2552 remain spaced apart at a proximal portion of sharp 2552. Sharp 2552 can be positioned in an off-center location during a molding process with hub components 2558, 2562, 2566, each of which may consist of a rigid plastic material.

FIGS. 11C and 11D are two side views depicting sharp module 2550 prior to assembly with sensor module 504 (FIG. 6B), and include sharp 2552, spacer 2568, hub push cylinder 2558, hub small cylinder 2562 and hub snap pawl 2566. In some embodiments, the relative distances between the sharp 2552 and hub components can be positioned as follows For example, distance, S₁, between the sharp 2552 and the radial center of hub can range from 0.50 mm to 1 mm (e.g., 0.89 mm). Height, S₂, of sharp spacer 2568 can range from 3 to 5 mm (e.g., 3.26 mm). Height, S₃, of hub can range from 5 to 10 mm (e.g., 6.77 mm). Length, S₄, of sharp 2552 can range from 1.5 mm to 25 mm (e.g., 8.55 mm), and may depend on the location of the insertion site on the subject.

FIG. 11E depicts a side cross-sectional side view of sharp module 2550, including sharp 2552, sharp spacer 2568 and hub components (hub snap pawl 2566, hub small cylinder 2562, and hub push cylinder 2558), as assembled with sensor module 504. As can be seen in FIG. 11E, sharp 2552 is positioned within sharp slot 2208 of sensor module 504 that includes a curved interior surface 2250, located at a distal end. Curved interior surface 2250 of sensor module 504 can be in contact with a portion of sharp 2552 and cause a deflection such that sharp distal tip 2556 is oriented toward central longitudinal axis 2545. As best seen in FIG. 11H, sharp 2552 can be positioned such that the distal portion and central longitudinal axis 2545 form an acute angle, S_{⊖}, that can range between 5° and 20°. In some embodiments, for example, S_{⊖}, can range from 5° to 17°, or 7° to 15°, or 9° to 13°, e.g., 9°, 10°, 11°, 12°, or 13°.

Referring still to FIG. 11E, near a distal end of sensor module 504 is protrusion 2251, which can enhance the perfusion of bodily fluid, such as dermal fluid. Although shown as a curved surface in FIG. 11E, protrusion 2251 can be shaped in any desired fashion. In addition, in some embodiments, multiple protrusions can be present. U.S. Patent Publication No. 2014/0275907, which is incorporated by reference herein in its entirety for all purposes, describes sensor devices having different protrusion configurations, each of which can be implemented with the embodiments described herein. Many of the embodiments described herein show the needle exiting from the protrusion, and in other embodiments, the needle can exit from the base of the sensor device adjacent the protrusion, and from that position extend over the tip of sensor 104.

Referring still to FIGS. 11E and 11F, sensor 104 can be a dermal sensor and can include sensor tail 2408, located at a distal end of sensor 104, and which can be positioned in a substantially parallel orientation to central longitudinal axis 2545. Distal end of sensor tail 2408 can be proximal to distal sharp tip 2556, either in a spaced relation with, at rest in, or at rest against a portion of sharp shaft 2554. As further depicted in FIG. 11E, sharp spacer 2568 provides a spaced relation between a proximal portion of sharp 2552 and sensor 104, such that the proximal portion of sharp 2552 and sensor 104 are not in contact. Sensor module 504 can further include sensor connector 2300 for housing a proximal portion of sensor 104 that is relatively perpendicular to a distal end of sensor 104.

FIG. 11F is a top-down cross-sectional view of sensor module 504. Sensor module 504 can include one or more sensor module snaps 2202 for coupling with a housing (not shown) of sensor control device 102. Sensor module 504 can also include sensor connector 2300, which can have sensor contacts 2302 for coupling with a proximal portion of sensor 104. Sensor connector 2300 can be made of silicone rubber that encapsulates compliant carbon impregnated polymer modules that serve as electrical conductive contacts 2302 between sensor 104 and electrical circuitry contacts for the electronics within sensor control device 102. The connector can also serve as a moisture barrier for sensor 104 when assembled in a compressed state after transfer from a container to an applicator and after application to a user's skin. Although three contacts 2302 are depicted, it should be understood that connector 2300 can have fewer contacts (e.g., two) or more contacts (e.g., four, five, six, etc.), depending on the particular type or configuration of sensor 104. Sensor connector 2300 can be further coupled with sensor module 504 by two connector posts 2206 positioned through a like number of apertures in connector 2300. Although two connector posts 2206 are depicted, it should be understood that any number of connector posts 2206 can be used to couple connector 2300 to sensor module 504.

FIGS. 11G and 11H are, respectively, a perspective view and a side view of another example embodiment of sharp module 2600 that can be used for the insertion of a dermal sensor. Sharp module 2600 is shown here prior to assembly with sensor module 504 (FIG. 6B), and can include components similar to those of the embodiments described with respect to FIGS. 11A and 11B, including sharp 2602, sharp shaft 2604, sharp distal tip 2606, hub push cylinder 2608, hub small cylinder 2612, hub snap pawl 2616 and hub snap pawl locating cylinder 2614. In some embodiments, sharp 2602 can be a “pre-bent” needle that includes a proximal portion 2603 that originates from a point external to sharp module 2600 and intersects, at an angle, a central point of the hub (e.g., through hub push cylinder 2608). Sharp 2602 can also include a distal portion 2605 that extends in a distal direction, at an angle, from a point near a distal portion of hub toward the insertion point of the user's skin. As shown in FIG. 11H, sharp 2602 can include an angled portion 2607 located external to hub push cylinder 2608, which can have a substantially 90° angle between proximal portion 2603 and distal portion 2605 of sharp 2602. Sharp module 2600 can also include a bend fin guide 2620 for maintaining “pre-bent” sharp 2602 in position during assembly and/or use, and can prevent lateral or rotational movement of sharp 2602 relative to hub components. Proximal portion 2603 of sharp 2602 can be “trimmed” from the hub after molding process is completed, and prior to assembly of sharp module 2600 with sensor module 504.

FIGS. 11I and 11J show, respectively, a side cross-sectional view and a side view of sharp module 2600 (including hub snap pawl 2616, hub small cylinder 2612, and hub push cylinder 2608), as assembled with sensor module 504. As can be seen in FIG. 11I, sensor module 504 includes sharp slot 2208, through which sharp 2602 can extend in an angled and distal direction. As described earlier, a proximal portion of sharp 2602 passes through bend fin guide 2620, which is coupled with a distal portion of sensor module 504. Sensor module 504 can also include sensor 104, which can be a dermal sensor. As seen in FIG. 11I, sharp 2602 and sensor tail 2408 can form an acute angle, S_{⊖}, at a point where their respective longitudinal axes converge. Angle S_{⊖} can range between 5° and 20°. In some embodiments, for example, S_{⊖}, can range from 5° to 17°, or 7° to 15°, or 9° to 13°, e.g., 9°, 10°, 11°, 12°, or 13°. In some embodiments, distal sharp tip 2606 is located at a distance, S₆, that is proximal to an end of sensor tail 2408. Distance, S₆, can range between 0.02 mm to 0.10 mm, e.g., 0.05 mm, 0.06 mm or 0.07 mm.

Referring still to FIGS. 11I and 11J, sensor module 504 can also include sensor connector 2300 for housing a proximal portion of sensor 104 that is relatively perpendicular to a distal end of sensor 104. Sensor module 504 can further include one or more sensor module snaps 2202 for coupling with a housing (not shown) of sensor control device 102. Sensor connector 2300 can include the same structures described with respect to FIG. 11F.

In the above embodiments, the sharp can be made of stainless steel or a like flexible material (e.g., material used to manufacture acupuncture needles), and dimensioned such that the applicator provides for insertion of at least a portion of the dermal sensor into the dermal layer, but not through the dermal layer of the skin. According to certain embodiments, the sharp has a cross sectional diameter (width) of from 0.1 mm to 0.5 mm. For example, the sharp may have a diameter of from 0.1 mm to 0.3 mm, such as from 0.15 mm to 0.25 mm, e.g., 0.16 mm to 0.22 mm in diameter. A given sharp may have a constant, i.e., uniform, width along its entire length, or may have a varying, i.e., changing, width along at least a portion of its length, such as the tip portion used to pierce the surface of the skin. For example, with respect to the embodiment shown in FIG. 111, width of sharp 2602 can narrow along a distal portion between bend fin guide 1620 and distal sharp tip 2606.

A sharp can also have a length to insert a dermal sensor just into the dermal layer, and no more. Insertion depth may be controlled by the length of the sharp, the configuration of the base and/or other applicator components that limit insertion depth. A sharp may have a length between 1.5 mm and 25 mm. For example, the sharp may have a length of from 1 mm to 3 mm, from 3 mm to S mm, from 5 mm to 7 mm, from 7 mm to 9 mm, from 9 mm to 11 mm, from 11 mm to 13 mm, from 13 mm to 15 mm, from 15 mm to 17 mm, from 17 mm to 19 mm, from 19 mm to 21 mm, from 21 mm to 23 mm, from 23 mm to 25 mm, or a length greater than 25 mm. It will be appreciated that while a sharp may have a length up to 25 mm, in certain embodiments the full length of the sharp is not inserted into the subject because it would extend beyond the dermal space. Non-inserted sharp length may provide for handling and manipulation of the sharp in an applicator set. Therefore, while a sharp may have a length up to 25 mm, the insertion depth of the sharp in the skin on a subject in those certain embodiments will be limited to the dermal layer, e.g., about 1.5 mm to 4 mm, depending on the skin location, as described in greater detail below. However, in all of the embodiments disclosed herein, the sharp can be configured to extend beyond the dermal space, such as into (or even fully through) subcutaneous tissue (e.g., 3 mm to 10 mm beneath the surface of the skin depending on the location of the skin on the body). Additionally, in some example embodiments, the sharps described herein can include hollow or partially hollow insertion needles, having an internal space or lumen. In other embodiments, however, the sharps described herein can include solid insertion needles, which do not have an internal space and/or lumen. Furthermore, a sharp of the subject applicator sets can also be bladed or non-bladed.

Likewise, in the above embodiments, a dermal sensor is sized so that at least a portion of the sensor is positioned in the dermal layer and no more, and a portion extends outside the skin in the transcutaneously positioned embodiments. That is, a dermal sensor is dimensioned such that when the dermal sensor is entirely or substantially entirely inserted into the dermal layer, the distal-most portion of the sensor (the insertion portion or insertion length) is positioned within the dermis of the subject and no portion of the sensor is inserted beyond a dermal layer of the subject when the sensor is operably dermally positioned.

The dimensions (e.g., the length) of the sensor may be selected according to the body site of the subject in which the sensor is to be inserted, as the depth and thickness of the epidermis and dermis exhibit a degree of variability depending on skin location. For example, the epidermis is only about 0.05 mm thick on the eyelids, but about 1.5 mm thick on the palms and the soles of the feet. The dermis is the thickest of the three layers of skin and ranges from about 1.5 mm to 4 mm thick, depending on the skin location. For implantation of the distal end of the sensor into, but not through, the dermal layer of the subject, the length of the inserted portion of the dermal sensor should be greater than the thickness of the epidermis, but should not exceed the combined thickness of the epidermis and dermis. Methods may include determining an insertion site on a body of a user and determining the depth of the dermal layer at the site, and selecting the appropriately-sized applicator set for the site.

In certain aspects, the sensor is an elongate sensor having a longest dimension (or “length”) of from 0.25 mm to 4 mm. The length of the sensor that is inserted, in the embodiments in which only a portion of a sensor is dermally inserted, ranges from 0.5 mm to 3 mm, such as from 1 mm to 2 mm, e.g., 1.5 mm. The dimensions of the sensor may also be expressed in terms of its aspect ratio. In certain embodiments, a dermal sensor has an aspect ratio of length to width (diameter) of about 30:1 to about 6:1. For example, the aspect ratio may be from about 25:1 to about 10:1, including 20:1 and 15:1. The inserted portion of a dermal sensor has sensing chemistry.

However, all of the embodiments disclosed herein can be configured such that at least a portion of the sensor is positioned beyond the dermal layer, such as into (or through) the subcutaneous tissue (or fat). For example, the sensor can be dimensioned such that when the sensor is entirely or substantially entirely inserted into the body, the distal-most portion of the sensor (the insertion portion or insertion length) is positioned within the subcutaneous tissue (beyond the dermis of the subject) and no portion of the sensor is inserted beyond the subcutaneous tissue of the subject when the sensor is operably positioned. As mentioned, the subcutaneous tissue is typically present in the region that is 3 mm to 10 mm beneath the outer skin surface, depending on the location of the skin on the body.

### Example Embodiments of Applicator Deployment

FIGS. 12A-12D are side cross-sectional views depicting an example embodiment of an applicator 150 during deployment of sensor control device 102, which can include a dermal sensor for sensing an analyte level in a dermal layer of the subject.

FIG. 12A shows applicator 150, prior to firing, in a state ready to be positioned against a subject's skin surface. Detent round 1404 of sheath 704 is positioned in “locked” groove 2332 in a locking rib of applicator housing 2702. Outer sharp carrier 3152 is coupled to inner spring 1106 and outer spring 1104, with both springs in a preloaded, compressed state. Outer sharp carrier 3152 is also retained by one or more sharp carrier lock arms 1524 of sensor electronics carrier 710. Sensor electronics carrier 710 is positioned within a proximal portion of sheath 704, wherein the inner diameter of sheath 704 is configured to deflect sharp carrier lock arms 1524 in an inward direction. A distal portion of outer sharp carrier 3152 is in contact with a proximally facing surface of sensor electronics carrier 710. Similarly, a distal portion of inner sharp carrier 3102 is coupled to a proximally facing surface of sensor electronics carrier 710. Sharp 2552 and sensor 104 are positioned within sheath 704.

In FIG. 12B, applicator 150 is shown in a “firing” state, where force applied to the proximal end of housing 2702 causes housing 2702 to move in a distal direction with respect to sheath 704. At this point, sharp 2552 and sensor 104 have extended from the distal end of sheath 704 and have already penetrated, or are in the process of penetrating, the subject's skin layer. Advancement of housing 2702 causes detent round 1404 to advance in a proximal direction relative to housing 2702 which, in turn, causes detent round 1404 to enter into a “free flight” state, in which detent round 1404 moves over firing surface 2337 with non-continuous contact or no contact. Sharp carrier lock arms 1524 of sensor electronics carrier 710 have also cleared the inner diameter of sheath 704 and are free to deflect outward into their biased position (indicated by outward arrows). Subsequently, sharp carrier lock arms 1524 disengage from outer sharp carrier 3152 which, in turn, begins to move in a proximal direction due to expansion of inner spring 1106 and outer spring 1104 (indicated by upward arrow). The expansion of inner spring 1106 also exerts a force in a distal direction causing inner sharp carrier 3102 to remain coupled to sensor electronics carrier 710. Similarly, the expansion of outer spring 1104 also exerts force in a distal direction securing sensor electronics carrier 710 in a distal position.

In FIG. 12C, outer sharp carrier 3152 continues to move in a proximal direction (indicated by upward arrow) due to continuing expansion of inner spring 1106 and outer spring 1104. After moving a predetermined distance in the proximal direction, outer carrier latch 3160 of outer sharp carrier 3152 engages inner carrier latch 3110 of inner sharp carrier 3102. As shown in FIG. 12C, sharp 2552 and sensor 104 remain in their respective positions due to the expansion forces in a distal direction created by springs 1104, 1106.

In FIG. 12D, inner sharp carrier 3102 is pulled in a proximal direction (indicated by elongated upward arrow) by force of outer carrier latch 3160. In turn, inner sharp carrier 3102 retracts sharp 2552 through sensor electronics carrier 710, leaving behind sensor 104 implanted in a dermal layer of the subject. Applicator 150 is shown in a “lockout” state, in which detent round 1404 of sheath 704 has advanced past the sheath stopping ramp (not shown) and within final lockout groove 2336 of housing 2702. As further shown in FIG. 12D, both inner sharp carrier 3102 and outer sharp carrier 3152 are fully retracted within applicator 150.

FIGS. 13A-13D are side cross-sectional views depicting an alternative embodiment of an applicator 151 during deployment of sensor control device 102 which can include a dermal sensor for sensing an analyte level in a dermal layer of the subject. Generally, applicator 151 operates in a similar manner as applicator 150, as described with respect to FIGS. 12A-12D, but additionally includes a retention mechanism to couple housing 3702 and sensor electronics carrier 2710. The retention mechanism operates to further increase the velocity of sharp insertion during firing, while delaying the sharp retraction sequence, as further described below.

As shown in FIG. 13A, applicator 151 is in a “locked” state, prior to firing. Sharp 2552 and sensor 104 are positioned within sheath 704, and applicator 151 is ready to be positioned against the subject's skin. Applicator housing 3702 includes heat stake post 1333 located on a distal portion of housing guide rib 3321. Heat stake post 1333 includes a flared end 1339, and protrudes from housing guide rib 3321 in a distal direction through aperture 1510 of sensor electronics carrier 2710. During the “locked” state, the proximally facing portion of sensor electronics carrier 2710 abuts against the proximal base of heat stake post 1333.

FIG. 13B shows applicator 151 in a “firing” state, wherein a force applied to the proximal end of housing 3702 causes housing 3702 to move in a distal direction with respect to sheath 704. Sharp 2552 and sensor 104 have extended from the distal end of sheath 704 and have already penetrated, or are in the process of penetrating, the subject's skin layer. Sharp carrier lock arms 1524, having cleared the inner diameter of sheath 704, deflect outward into their biased positions (indicated by outward arrows), and disengage from outer sharp carrier 3152. Outer sharp carrier 3152, in turn, begins to move in a proximal direction due to expansion of inner spring 1106 and outer spring 1104. Expansion of inner spring 1106 and outer spring 1104, as well as the movement of outer sharp carrier 3152 in a proximal direction, creates a corresponding opposing force in a distal direction against inner sharp carrier 3102 and sensor electronics carrier 2710 (indicated by downward arrow). This force causes inner sharp carrier 3102 and sensor electronics carrier 2710 to further advance in a distal direction along heat stake post 1333, thereby increasing the velocity of the sharp in a distal direction during insertion. At this point, inner sharp carrier 3102 and sensor electronics carrier 2710 remain coupled.

In FIG. 13C, outer sharp carrier 3152 continues to move in a proximal direction (indicated by upward arrow) due to continuing expansion of inner spring 1106 and outer spring 1104. Sensor electronics carrier 2710 has advanced in a distal direction along heat stake post 1333 until it reaches flared end 1339 of the post. Flared end 1339, which is larger than aperture 1510, abuts against ledges 1513 in sensor electronics carrier 2710, thereby preventing sensor electronics carrier 2710 from completely disengaging from housing guide rib 3321 of housing 3702. After moving a predetermined distance in the proximal direction, outer carrier latch 3160 (not shown) of outer sharp carrier 3152 engages inner carrier latch 3110 (not shown) of inner sharp carrier 3102 (in circled area ‘K’). Sharp 2552 and sensor 104 remain in an extended state outside of sheath 704.

FIG. 13D shows applicator 151 in the “lockout” state. The continuing expansion of inner spring 1106 and outer spring 1104 cause outer sharp carrier 3152 to further advance in a proximal direction. Subsequently, outer carrier latch 3160 (not shown) engages with inner sharp carrier 3102 and pulls inner sharp carrier 3102 in a proximal direction (indicated by elongated upward arrow). In turn, inner sharp carrier 3102 retracts sharp 2552 through sensor electronics carrier 2710, leaving behind sensor 104 implanted in a dermal layer of the subject. Detent round 1404 of sheath 704 is positioned in the final lockout groove 2336, and both inner sharp carrier 3102 and outer sharp carrier 3152 are fully retracted within applicator 151.

With regard to the embodiments in FIGS. 13A-13D, heat stake post 1333 is described as a retention mechanism to couple housing 3702 and sensor electronics carrier 2710. It should be understood, however, that different retention mechanisms may be utilized, such as snap-in arms 1329, as described with respect to FIG. 9E, snaps, hooks, ball locks, latches, pins and/or other like retaining devices and structures.

With regard to the embodiments in FIGS. 12A-12D and 13A-13D, a sharp carrier assembly including an inner spring for maintaining the position of the inner sharp carrier is described. It will be understood by those of skill in the art that other devices and mechanisms for maintaining the position of the inner sharp carrier are fully within the scope of the disclosed embodiments. For example, an inner sharp carrier detent for engaging with the sensor electronics carrier (as described with respect to FIG. 10D), inner sharp carrier having one or more locking nubs for engaging with the sensor electronics carrier (as described with respect to FIG. 10E), as well as snaps, hooks, ball locks, latches, pins, and screw threads, can be used individually or in combination to retain inner sharp carrier in position during the “firing” sequence of the applicator.

FIGS. 14A-14C are side cross-sectional views depicting another alternative embodiment of applicator 152 during deployment of sensor control device 102. As with the previous embodiments, applicator 152 is initially positioned against the subject's skin and a force is applied to the proximal end of housing 7702, causing housing 7702 to move in a distal direction with respect to sheath 6704. Thereafter, sharp 2552 and sensor 104 extend from the distal end of sheath 6704 and penetrate the subject's skin layer. Unlike the previous embodiments (FIGS. 12A-12D and 13A-13D), however, applicator 152 utilizes a motion- actuated sharp retraction mechanism which, as described in further detail below, retracts the sharp when the user moves applicator 152 away from the skin.

FIG. 14A shows applicator 152 in an early “lockout” state, after detent round 1404 of sheath 6704 has advanced over sloped firing surface 7338, by virtue of the user applying a first force upon the applicator, and reached two-way lockout recess 7336. At this stage, sharp 2252 has penetrated the skin layer and sensor 104 has been inserted into the dermal layer. Furthermore, as best seen in call-out 14A-1, one or more sharp carrier lock arms 6524 of sensor electronics carrier 6710 are biased in an outward direction and pushed against one or more corresponding carrier arm ramps 6415 of sheath 6704. In this position, carrier arm ramps 6415 impart a downward pushing force on lock arms 6524, thereby constraining sharp carrier 1102 against sensor electronics carrier 6710. Additionally, as seen in call-out 14A-2, snap-in arms 1329 of housing 7702 protrude through aperture 1510 of sensor electronics carrier 6710. At this stage, the distal edge of the housing is flush against aperture 1510 and aperture ledge 1513 of sensor electronics carrier 6710.

FIG. 14B shows applicator 152, after the “lockout” state, as the user begins to move applicator 152 away from the skin by applying a second force to applicator 152. The second force, which can be in a proximal or “upward” direction, for example, can be in the opposite direction as the first force, which can be in a distal or “downward” direction. An adhesive layer (not shown) on the bottom surface of sensor control device 102 keeps sensor control device 102 against the subject's skin, and movement of applicator 152 in a proximal direction results in a pull force on the sensor electronics carrier 6710 relative to housing 7702. As best seen in call-out 14B-1, carrier arm ramps 6415 include a beveled end surface which imparts a force in a distal direction onto lock arms 6524, and causes sensor electronics carrier 6710 to separate from housing 7702. Consequently, as shown in call-out 14B-2, sensor electronics carrier 6710 moves in a distal direction (i.e., towards the skin) relative to housing 7702, as aperture ledge 1513 moves closer to snap-in detents 1331 of snap-in arms 1329.

FIG. 14C shows applicator 152 as it is pulled away from the skin. As seen in call-out 14C-1, lock arms 6524 have cleared the carrier arm ramps 6415. Subsequently, sharp carrier 1102 is released and moves in a proximal direction from the force of compression spring 1104, thereby retracting sharp 2252. Also, as shown in call-out 14C-2, as snap-in detents 1331 of snap-in arms 1329 abut against ledge 1513, sensor electronics carrier 6710 can move no further away from housing 7702. Subsequently, as user pulls applicator 152 away from the skin, sensor control device 102 separates from sensor electronics carrier 6710 and is now attached to skin with sensor 104 inserted.

FIGS. 15A-15B are, respectively, a side cross-sectional view and a perspective cross-sectional view, both depicting another alternative embodiment of an applicator 153 during deployment of sensor control device 102. Applicator 153 also utilizes a motion-actuated sharp retraction mechanism and generally operates in a similar manner to applicator 152, as described with respect to FIGS. 14A-14C.

Turning to FIG. 15A, applicator 153 is shown in a state prior to firing, ready to be positioned against a subject's skin surface. Detent round 1404 of sheath 6704 is positioned in “locked” groove 6332 of locking rib 6340 in housing 6702. In addition, locking rib 6340 includes a sloped firing surface 6338 which creates a downward force on sheath 6704 during firing. Sheath 6704 also includes inner sheath ribs 6425 disposed on the inner surface of sheath 6704. As previously described with respect to FIGS. 8F-8H, the interfaces between inner sheath ribs 6425 and rib notches (not shown) of sensor electronics carrier 6710 maintain the axial alignment of the sheath 6704 and sensor electronics carrier 6710, and further prevent unwanted rotational and/or lateral movement during the sensor insertion process.

Referring still to FIG. 15A, sharp carrier 1102 is coupled with compression spring 1104, which is in a preloaded, compressed state. Sharp carrier 1102 is retained by one or more carrier lock arms 6524 of sensor electronics carrier 6710. Prior to firing, sharp 2552 and sensor 104 are positioned within sheath 6704.

Turning to FIG. 15B, applicator 153 is shown in an early “lockout” state, after sensor 104 has been inserted, but before sharp 2552 has been retracted. Detent round 1404 has advanced over sloped firing surface 6338 and reached the final lockout recess 6336 in locking rib 6340, which prevents further movement of sheath 6704 in a distal direction relative to housing 6702. In addition, sheath 6704 includes a sheath travel limiter ledge 6720 which, in the “lockout” state, abuts against a bottom edge 6331 of housing 6702, thereby preventing further movement of sheath 6704 in a proximal direction relative to housing 6702. Thus, in the “lockout” state, sheath 6704 can be prevented from further traveling in either a proximal or distal direction relative to housing 6702. In addition, at this stage, carrier lock arms 6524 have not cleared ramps 6415 of sheath 6704, and ledge 1513 of sensor electronics carrier 6710 is flush against housing 6702. Thus, the motion-actuated sharp retraction mechanism has not yet been initiated. Subsequently, as the user pulls away applicator 153 from the skin, carrier lock arms 6524 will clear ramps 6415, thereby releasing sharp carrier 1102 and initiating the sharp retraction mechanism (as described with respect to FIG. 14C).

With respect to the embodiments in FIGS. 14A-14C and 15A-15B, it should be understood that embodiments, such as applicators 152 and 153, can generally have a slower effective speed of insertion compared to applicators shown in FIGS. 12A-12D and 13A-13D. In addition, sheath 6704 of FIGS. 14A-14C and 15A-15B can be of shorter length than the sheaths depicted with respect to FIGS. 12A-12D and 13A-13D. Furthermore, in some embodiments, sheath 6704 can also include a base surface coated with an adhesive for adhering to the skin surface of the user.

FIGS. 16A-16C are side cross-sectional views depicting another alternative example embodiment of applicator 154 during deployment of sensor control device 102. As with previous embodiments, applicator 154 is initially positioned against the subject's skin and a force is applied to the proximal end of housing 702, causing housing 702 to move in a distal direction with respect to sheath 8704. Thereafter, sharp 2552 and sensor 104 extend from the distal end of sheath 8704 and penetrate the subject's skin layer. According to one aspect of the disclosed embodiments, applicator 154 can include a dual-stage needle retraction mechanism, in which sharp 2252 is partially retracted at a first stage to minimize further penetration by sharp 2552 into the subject, while sensor 104 can further penetrate the tissue, e.g., the dermis or the subcutaneous tissue, to its final position. As further described below, in many embodiments, the dual-stage needle retraction mechanism can be implemented by a plurality of slots, including a sheath slot 8706 and sharp carrier slot 8104 (as depicted in FIG. 10F), each of which can be configured to receive at least a portion of a sharp carrier lock arm 1524 of sensor electronics carrier 710.

Referring first to FIG. 16A, applicator 154 is shown in a “locked” state, prior to firing, in which applicator 154 is ready to be positioned against a subject's skin surface. Sharp 2552 and sensor 104 are positioned within sheath 8704. Sensor electronics carrier 710 is resting radially against the inner diameter of sheath 8704.

FIG. 16B shows applicator 154 after a force has been applied to the proximal end of housing 702, causing housing 702 to move in a distal direction with respect to sheath 8704. Sharp 2552 and sensor 104 have extended from the distal end of sheath 8704, and have already penetrated, or are in the process of penetrating, the subject's skin layer. As sheath 8704 moves in a proximal direction relative to housing 702 and sensor electronics carrier 710, at least a portion of each sharp carrier lock arm 1524 of sensor electronics carrier 710 can be received into a sharp carrier slot 8104 disposed on sharp carrier 8102 and a sheath slot 8706 disposed on sheath 8704. (See also FIG. 10F.) As a portion of each lock arm 1524 is received into slots 8104 and 8706, lock arm 1524 can partially deflect in an outward direction, allowing sharp carrier 8102 to move a limited distance in a proximal direction due to the force of expansion of preloaded compression spring 1104 in sharp carrier 8102. In this manner, according to one aspect of the embodiments, sharp 2552 can be partially retracted, or maintained in a stationary position relative to the skin surface, during or after the first stage of the dual-stage needle retraction process. In addition, according to another aspect of the embodiments, during the first stage of the dual-stage sharp retraction, a distal portion of sensor 104 can continue to penetrate the tissue, e.g., the dermis or the subcutaneous tissue of the subject, while a proximal portion of sensor 104 can remain within sharp 2552.

FIG. 16C shows applicator 154 at the second stage of the dual-stage needle retraction process. As housing 702 continues to move in a distal direction with respect to sheath 8704, sharp carrier lock arms 1524 of sensor electronics carrier 710 have cleared the inner diameter of sheath 8704, and are free to deflect outward into their biased position. Subsequently, sharp carrier lock arms 1524 disengage from sharp carrier 8102 which, in turn, moves further in a proximal direction due to further expansion of spring 1104, thereby causing sharp 2552 to further retract into applicator 154. As can also be seen in FIG. 16C, applicator 154 is shown in a “lockout” state, in which detent round 1404 of sheath 8704 has advanced past the sheath stopping ramp 1338 and within final lockout recess 1336 of housing 702.

With respect to the embodiments in FIGS. 16A-16C, those of skill in the art will appreciate that embodiments having a dual-stage needle retraction mechanism, such as applicator 154, can be configured to reduce the depth of penetration by sharp 2252 relative to, for example, the sensor tip. In this manner, these embodiments can reduce early sensor attenuation or sensor inaccuracy during the first few hours after insertion, which can be caused by trauma at the insertion site. Furthermore, although sharp carrier slot 8104 and sheath slot 8706 are depicted at certain positions along sharp carrier 8102 and sheath 8704, respectively, those of skill in the art will appreciate that other positions along the sharp carrier 8102 and/or sheath 8704, configurations (e.g., three, four or five slots) and/or geometries (e.g, angled surfaces, curved surfaces, concave surfaces, etc.) which are adapted to cause a partial release of the sharp carrier lock arms are fully within the scope of the present disclosure In some embodiments, for example, the height of sheath slot 8706 in sheath 8704 can be varied to change the timing of the retraction relative to how far sheath 8704 has been retracted. Similarly, in other embodiments, the height of sharp carrier slot 8104 can be varied to change the distance of the partial retraction of sharp 2552.

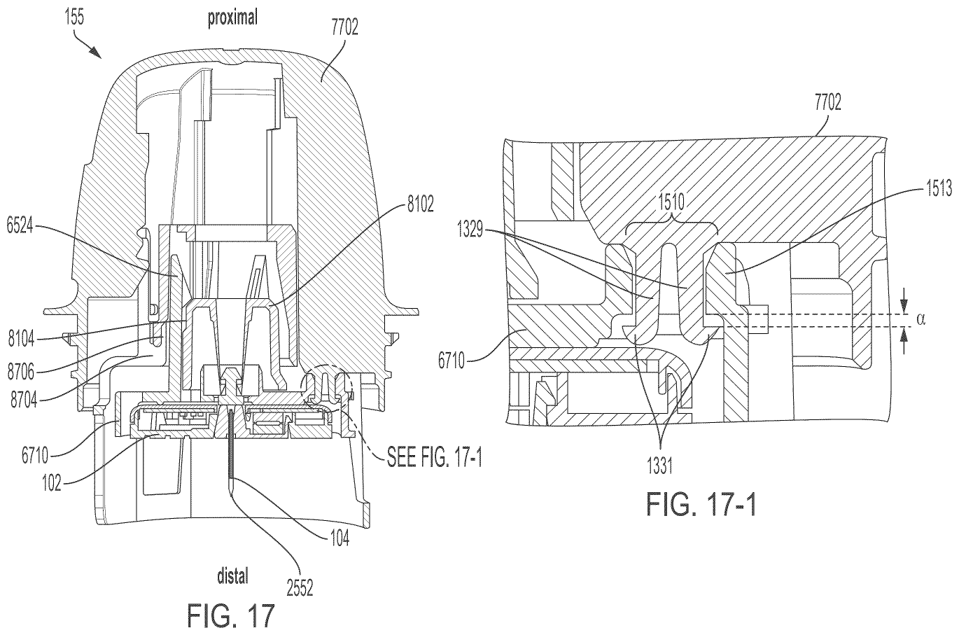

Turning to FIG. 17, a side cross-sectional view of another example alternative embodiment is provided, with applicator 155 shown ready for use in an “armed” position. According to one aspect of the embodiments, applicator 155 can include a compliant dual-stage needle retraction mechanism which can operate in a similar manner to the embodiments described with respect to FIGS. 16A-C. In many embodiments, for example, applicator 155 can include a sharp carrier slot 8104 of sharp carrier 8102 and a sheath slot 8706 of sheath 8704, each of which can be configured to receive at least a portion of lock arms 6524 of sensor electronics carrier 6710. During operation, as a portion of each lock arm 6524 is received into slots 8104 and 8706, lock arm 6524 can partially deflect in an outward direction, allowing sharp carrier 8102 to move a limited distance in a proximal direction due to the force of expansion of a preloaded compression spring (not shown) disposed in sharp carrier 8102. In this manner, according to one aspect of the embodiments, sharp 2552 can be partially retracted, or maintained in a stationary position relative to the skin surface, during or after the first stage of the dual-stage needle retraction process, while a distal portion of sensor 104 can continue to penetrate the tissue, e.g., the dermis or the subcutaneous tissue. As housing 7702 continues to move in a distal direction, the second stage of the dual-stage needle retraction mechanism is initiated In particular, lock arms 6524 can clear the inner diameter of sheath 8704 and deflect outward into their biased position, thereby disengaging from sharp carrier 8102, which, in turn, moves further in a proximal direction due to further expansion of the spring, and retracts sharp 2552 into applicator 155.

Referring still to FIG. 17, according to another aspect of the embodiments, applicator 155 can include a compliance mechanism between sensor electronics carrier 6710 and housing 7702. In some embodiments, as best seen in call-out 17-1 of FIG. 17, housing 7702 of applicator 155 can include one or more snap-in arms 1329, which can protrude through aperture 1510 of sensor electronics carrier 6710. At a distal portion of snap-in arms 1329, one or more snap-in detents 1331 can prevent snap-in arms 1329 from disengaging from sensor electronics carrier 6710. Furthermore, as seen in call-out 17-1 of FIG. 17, the bottom edge of aperture ledge 1513 and the one or more snap-in detents 1331 are in a spaced relation by a predetermined amount of clearance, α, which can allow for limited movement by, collectively, sheath 8704, sharp carrier 8102, sensor electronics carrier 6710, and sensor control unit 102 relative to housing 7702.

According to one aspect of the embodiments, the predetermined clearance, a, can allow for gimbaling by sensor electronics carrier 6710 relative to housing 7702 which, in turn, can cause an angular displacement of sharp 2552 and sensor 104 relative to housing 7702 during insertion. For example, when applicator 155 is in the “armed” position, as shown in FIG. 17, a distal portion of analyte sensor 104 and a longitudinal axis 8545 of housing 7702 are substantially parallel to each other. According to one aspect of the embodiments, as force is applied to the housing 7702 and applicator 155 is fired, sensor electronics carrier 6710 can gimbal in relation to housing 7702 and cause the distal portion of analyte sensor 104 and the longitudinal axis 8545 to be in a non-parallel relation. In this regard, sharp 2552 and sensor 104 can follow a path of least resistance through the tissue, rather than being forced in the same axial direction as housing 7702, which, in turn, can reduce trauma to tissue during penetration and reduce early signal attenuation or sensor inaccuracy during the first few hours after insertion.

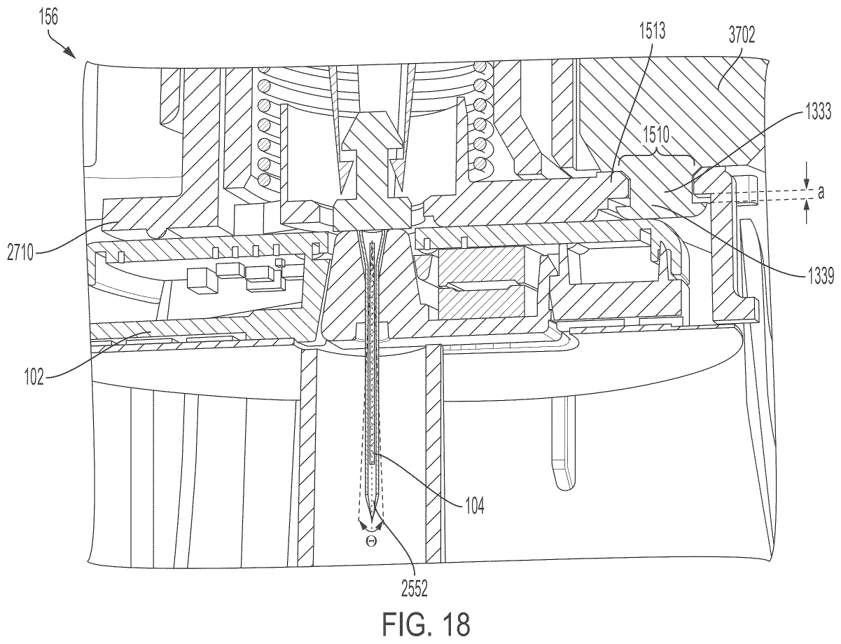

FIG. 18 is a partial cross-sectional view of another example embodiment of an applicator 156, also having a compliance mechanism. According to one aspect of some embodiments, housing 3702 of applicator 156 can include a heat stake post 1333, which can protrude through aperture 1510 of sensor electronics carrier 2710. Heat stake post 1333 can have a flared distal end 1339, which can be configured to prevent heat stake post 1333 from disengaging from sensor electronics carrier 2710. Furthermore, like the embodiments described with respect to FIG. 17, the bottom edge of aperture ledge 1513 and flared distal end 1339 of heat stake post 1333 can be in a spaced relation by a predetermined amount of clearance, α, which can allow for limited freedom of movement by sensor electronics carrier 2710.

According to another aspect of the embodiments, predetermined clearance, α, can allow for gimballing movement by the sheath, sensor electronics carrier 2710, and sensor control unit 102 relative to housing 3702, as well as angular displacement of sharp 2552 and sensor 104 during insertion. Referring still to FIG. 18, the degree and range of angular displacement, ⊖, by sharp 2552 and sensor 104 can be a function of the amount of the predetermined clearance, α. In some embodiments, for example, a predetermined clearance, α, of 0.5 millimeters can result in an angular displacement of approximately 2 degrees and 0.6 millimeters. Those of skill in the art will recognize that these measurements are provided solely for the purpose of illustration, and are in no way meant to limit the predetermined clearance or angular displacement to any particular value or range of values.

With respect to the embodiments in FIGS. 17 and 18, although some embodiments including the compliance mechanism are described in combination with the dual-stage needle retraction mechanism, it will be understood by those of skill in the art that the compliance mechanism can be combined with applicators having other types of retraction mechanisms, such as those embodiments described with respect to FIGS. 12A-12D, 13A-13D, 14A-14C, and 15A-15B, as well as applicators described in U.S. Patent Publication No. 2013/0150691 and U.S. Patent Publication No. 2016/0331283, which are incorporated by reference herein in its entirety for all purposes.

With respect to the embodiments in FIGS. 12A-12D, 13A-13D, 14A-14C, 15A-15B, 16A-16C, 17 and 18, although sharp 2552 is described, it should be understood that any of the sharps, sharp modules and sensor modules described herein with respect to FIGS. 11A-11J can be used.

With respect to any of the applicator embodiments in FIGS. 12A-12D, 13A-13D, 14A-14C, 15A-15B, 16A-16C, 17, and 18, as well as any of the components thereof, including but not limited to the sharp, sharp module and sensor module embodiments of FIGS. 11A-11J, those of skill in the art will understand that said embodiments can be dimensioned and configured for use with sensors configured to sense an analyte level in a bodily fluid in the epidermis, dermis, or subcutaneous tissue of a subject. In some embodiments, for example, sharps and distal portions of analyte sensors disclosed herein can both be dimensioned and configured to be positioned at a particular end-depth (i.e., the furthest point of penetration in a tissue or layer of the subject's body, e.g., in the epidermis, dermis, or subcutaneous tissue). With respect to some applicator embodiments, e.g., in embodiments having a dual-stage needle retraction mechanism, those of skill in the art will appreciate that certain embodiments of sharps can be dimensioned and configured to be positioned at a different end-depth in the subject's body relative to the final end-depth of the analyte sensor. In some embodiments, for example, a sharp can be positioned at a first end-depth in the subject's epidermis prior to retraction, while a distal portion of an analyte sensor can be positioned at a second end-depth in the subject's dermis. In other embodiments, a sharp can be positioned at a first end-depth in the subject's dermis prior to retraction, while a distal portion of an analyte sensor can be positioned at a second end-depth in the subject's subcutaneous tissue. In still other embodiments, a sharp can be positioned at a first end-depth prior to retraction and the analyte sensor can be positioned at a second end-depth, wherein the first end-depth and second end-depths are both in the same layer or tissue of the subject's body.

A number of deflectable structures are described herein, including but not limited to deflectable detent snaps 1402, deflectable locking arms 1412, sharp carrier lock arms 1524, sharp retention arms 1618, and module snaps 2202. These deflectable structures are composed of a resilient material such as plastic or metal (or others) and operate in a manner well known to those of ordinary skill in the art. The deflectable structures each has a resting state or position that the resilient material is biased towards. If a force is applied that causes the structure to deflect or move from this resting state or position, then the bias of the resilient material will cause the structure to return to the resting state or position once the force is removed (or lessened). In many instances these structures are configured as arms with detents, or snaps, but other structures or configurations can be used that retain the same characteristics of deflectability and ability to return to a resting position, including but not limited to a leg, a clip, a catch, an abutment on a deflectable member, and the like.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1-150. (canceled)

151. An applicator assembly for inserting an in vivo glucose sensor in a subject, the applicator assembly comprising:
(1) a sharp carrier subassembly, comprising:
a sharp comprising a sharp hub and a sharp distal tip;
a plurality of proximally-extending sharp retention arms configured to retain the sharp hub; and
one or more locking nubs protruding in an outward direction away from a central longitudinal axis of the sharp carrier subassembly;
(2) a sensor electronics carrier configured to retain a sensor control device, the sensor electronics carrier comprising one or more carrier nub slots, wherein the one or more locking nubs of the sharp carrier subassembly are configured to be in fitted contact with the one or more carrier nub slots of the sensor electronics carrier;
(3) the sensor control device, comprising sensor electronics and the in vivo glucose sensor, the in vivo glucose sensor comprising:
a proximal portion coupled with the sensor electronics; and
a distal portion configured to be positioned under a skin surface of the subject and in contact with a bodily fluid of the subject; and
wherein the sharp carrier subassembly, the sensor electronics carrier, and the sensor control device are configured to advance from a proximal position to a distal position,
wherein the sharp carrier subassembly is configured to rotate in response to a force applied along the central longitudinal axis of the sharp carrier subassembly,
wherein each of the one or more locking nubs is configured to slidably advance toward an open end of a corresponding each of the one or more carrier nub slots during rotation of the sharp carrier assembly, and
wherein advancement of the each of the one or more locking nubs toward the open end causes the sharp carrier subassembly to disengage from the sensor electronics carrier and retract the sharp to a retracted position.

152. The applicator assembly of claim 151, wherein the each of the one or more locking nubs is disposed on an outer cylindrical surface of the sharp carrier subassembly.

153. The applicator assembly of claim 152, wherein the each of the one or more locking nubs is further disposed on a proximal portion of the sharp carrier subassembly.

154. The applicator assembly of claim 151, wherein the corresponding each of the one or more carrier nub slots comprises a cut-out in the sensor electronics carrier.

155. The applicator assembly of claim 154, wherein the cut-out comprises a first slot portion having the open end, and a second slot portion that is substantially perpendicular with the first portion.

156. The applicator assembly of claim 151, wherein the open end of the corresponding each of the one or more carrier nub slots is disposed on a proximal portion of the sensor electronics carrier.

157. The applicator assembly of claim 154, wherein the cut-out comprises an L-shaped cut.

158. The applicator assembly of claim 151, further comprising a retraction spring, wherein the retraction spring comprises:
a proximal end coupled with a distally-facing surface of the sharp carrier subassembly; and
a distal end coupled with a proximally-facing surface of the sensor electronics carrier.

159. The applicator assembly of claim 158, wherein the retraction spring is in a preloaded and compressed state prior to the advancement of the each of the one or more locking nubs toward the open end.

160. The applicator assembly of claim 158, wherein the retraction spring is configured to apply a force to the distally-facing surface of the sharp carrier subassembly to cause retraction of the sharp to the retracted position.

161. The applicator assembly of claim 151, wherein each of the proximally-extending sharp retention arms of the plurality of proximally-extending sharp retention arms comprises a distal end having a sharp retention clip.

162. The applicator assembly of claim 161, wherein the sharp retention clip comprises a proximal surface that is substantially perpendicular to the central longitudinal axis of the sharp carrier subassembly.

163. The applicator assembly of claim 162, wherein the proximal surface is configured to abut a distally facing surface of the sharp hub.

164. The applicator assembly of claim 151, further comprising a sheath comprising a distal end for placement on the skin surface of the subject.

165. The applicator assembly of claim 164, further comprising an applicator housing configured to receive a manual force along the central longitudinal axis of the sharp carrier subassembly in a distal direction.

166. The applicator assembly of claim 165, wherein the housing and the sheath are configured such that the manual force causes the housing to advance in the distal direction relative to the sheath while the sheath remains on the skin surface of the subject.

167. The applicator assembly of claim 151, wherein the sensor electronics comprises one or more processors, memory, wireless communication circuitry, and a power supply.

168. The applicator assembly of claim 167, wherein the wireless communication circuitry is configured to transmit data indicative of the subject's glucose levels according to a Bluetooth or Bluetooth Low Energy communication protocol.

169. The applicator assembly of claim 151, wherein the sharp carrier subassembly, the sensor electronics carrier, and the sensor control device are further configured to advance along a linear path from the proximal position to the distal position.

170. The applicator assembly of claim 164, wherein the sheath is configured to remain on the skin surface of the subject while the distal portion of the in vivo glucose sensor is positioned under the skin surface of the subject.
